(19) United States
(12) Patent Application Publication
(10) Pub. No.: US 2019/0119639 A1
(43) Pub. Date: Apr. 25, 2019

(54) CELL COMPOSITIONS COMPRISING ANTIGEN-SPECIFIC T CELLS FOR ADOPTIVE THERAPY

(71) Applicant: Nexlmmune, Inc., Gaithersburg, MD (US)

(72) Inventors: Mathias OELKE, Gaithersburg, MD (US); Kristi JONES, Gaithersburg, MD (US); Sojung KIM, Gaithersburg, MD (US); Lauren SUAREZ, Gaithersburg, MD (US); Ken CARTER, Gaithersburg, MD (US); Scott CARMER, Gaithersburg, MD (US); Dan BEDNARIK, Gaithersburg, MD (US)

(21) Appl. No.: 16/136,931

(22) Filed: Sep. 20, 2018

Related U.S. Application Data
(60) Provisional application No. 62/656,679, filed on Apr. 12, 2018.
(60) Provisional application No. 62/561,044, filed on Sep. 20, 2017.

(51) Int. Cl. C12N 5/0783; A61K 35/17; A61P 35/02

(52) U.S. Cl. CPC C12N 5/0636; A61K 35/17; A61P 35/02; C12N 2527/00; C12N 2501/21; C12N 2501/2301; C12N 2501/2321; C12N 2501/231; C12N 2501/2304; C12N 2501/2302; C12N 2501/24; C12N 2502/1114; C12N 2501/2306

(57) ABSTRACT

The present invention provides an isolated cell composition suitable for adoptive immunotherapy, as well as methods of manufacturing the cell compositions and methods of treatment with the cell compositions. The composition comprises, in a pharmaceutically acceptable carrier, at least about 10⁶ CD8+ T cells specific for target peptide antigen(s). In various embodiments, the composition is predominately CD8+ T cells, and at least about 20% of T cells in the composition exhibit a central or effector memory phenotype, providing for a robust and durable adoptive therapy from a natural T cell repertoire that has undergone natural selection.

### PRIORITY

This application claims the benefit of U.S. Provisional Application No. 62/561,044, filed Sep. 20, 2017, and the benefit of U.S. Provisional Application No. 62/656,679, filed Apr. 12, 2018, each which is incorporated herein by reference in its entirety.

### BACKGROUND

Adoptive immunotherapies, such as donor lymphocyte infusions, are used for the treatment of leukemia relapse post hematopoietic stem-cell transplantation (HSCT) to enhance the graft versus leukemia (GVL) effect. These approaches often take several months to take effect; and require very large doses of cells, which results in a substantial risk of graft versus host disease (GVHD). See, McLaughlin L, et al., Adoptive T-cell therapies for refractory/relapsed leukemia and lymphoma; current strategies and recent advances. Ther. Adv. Hematol. 2015 Vol. 6(6) 295-307.

With current therapeutic options, the outcome for leukemia patients who relapse e.g., after HSCT, is bleak. While adoptive cell therapies can provide some benefit, the numbers of target-specific cells that can be provided are often insufficient and highly variable, and it is difficult to activate and expand naive T cell populations from donor lymphocytes ex vivo, especially with regard to cancer-specific CTL precursors that are often extremely low and even undetectable in peripheral blood of healthy individuals. Quintarelli C, et al., Cytotoxic T lymphocytes directed to the preferentially expressed antigens of melanoma (PRAME) target chronic myeloid leukemia. Blood 2008; 112: 1876-1885. Further, cell therapies such as chimeric antigen receptor (CAR) T cells and natural killer cell therapies tend to induce exhausted cell phenotypes that are not sufficiently robust and/or have limited persistence in vivo, and can exhibit on target off-tissue toxicities. See, Cruz and Bollard, T-cell and natural killer cell therapies for hematological malignancies after hematopoietic stem cell transplantation; enhancing the graft-versus-leukemia effect. Haematologica 2015; 100(6) 709-719. Further, these therapies generally have limited flexibility due to the engineered single target.

Cell compositions are needed to provide for more effective and safer adoptive immunotherapy options, including for patients suffering from leukemia or lymphoma (including acute or chronic leukemia), as well as other patients that could benefit from adoptive immunotherapy. In various aspects and embodiments, the present invention addresses these needs.

### SUMMARY OF THE INVENTION

In various aspects and embodiments, the invention provides an isolated cell composition suitable for adoptive immunotherapy, as well as methods of manufacturing the cell compositions and methods of treatment with the cell compositions. The composition comprises, in a pharmaceutically acceptable carrier, at least about 10⁶ CD8+ T cells specific for target peptide antigen(s). In various embodiments, the composition is predominately CD8+ T cells, and at least about 20% of T cells in the composition exhibit a central or effector memory phenotype, providing for a robust and durable adoptive therapy from a natural T cell repertoire that has undergone natural selection. The cell composition does not comprise T cells expressing a chimeric antigen receptor or a recombinant TCR, and therefore, in various embodiments, provides an alternative to these technologies that often produce more exhausted T cell phenotypes and less durable responses and greater toxicities.

In various embodiments, the cell composition comprises at least about 10⁷ CD8+ T cells specific for the target peptide antigens, or at least about 10⁸, at least about 10⁹, or at least about 10¹⁰ CD8+ T cells specific for the target peptide antigens, to provide robust destruction of target cells and a long persistence in vivo. For example, for treatment of acute myelogenous leukemia (AML) or myelodysplastic syndrome, the cell composition may comprise T cells specific for WT1, PRAME, Survivin, and Cyclin A1 peptide antigens.

In various embodiments, the T cells in the composition (and/or the T cells specific for the target antigens) are at least about 50% central or effector memory T cells, or in some embodiments are at least about 70% central or effector memory cells, or at least about 80% central or effector memory T cells. In some embodiments, the memory cells are from about 25:75 to about 75:25 central to effector memory cells. The cell composition comprises less than about 20% terminally differentiated memory T cells (e.g., Tₑₘᵣₐ cells), and no more than about 20% naive cells. In some embodiments, the cell composition comprises from about 5 to about 25% T memory stem cells (T_{SCM}). This cell phenotype can be created and/or controlled using an enrichment and expansion process with paramagnetic artificial Antigen Presenting Cells (aAPCs) and a recombinant T cell growth factor cocktail.

In various embodiments, the cell composition is at least 90% CD8+ T cells (e.g., CD3+CD8+ cells). For example, the isolated cell composition may be characterized by having less than about 10%, or less than about 5% CD4+ T cells. When expanding CD8+ T cells ex vivo, CD4+ cells have a tendency to overgrow the CD8+ cells and compete for growth signals, and are not necessary for a robust and durable in vivo response.

In various embodiments, the antigen-specific T cells display a polyfunctional phenotype upon activation. For example, upon activation the T cells are positive for two or more of: intracellular staining for IL-2, IFN-γ production, production of TNF-α, and CD107A. In various embodiments, at least 50%, or at least 70%, of the antigen-specific T cells display at least two of these markers. In various embodiments, at least 50% or at least 70% of the antigen-specific T cells display at least three of these markers, or in some embodiments all four of these markers.

Cell compositions in accordance with various embodiments can be prepared by an enrichment and expansion process. In some embodiments, CD8+ cells are enriched that are specific for the target antigen(s) (e.g., tumor associated antigens or viral-associated antigens). This cell population, even when predominately naive cells in the source lymphocytes, can be rapidly expanded in culture to arrive at the cell compositions described herein. Enrichment can take place using paramagnetic beads to positively select cell populations, and which can have the added advantage of activating naive cells due to potent magnetic clustering of T cell surface receptors. For example, paramagnetic beads or nanoparticles may contain monomeric or multimeric (e.g., dimeric) HLA ligands presenting peptide antigens, along with a co-stimulation signal on the same or different particles, such as an agonist for CD28 (e.g., an antibody agonist of CD28). In some embodiments, CD28+ cells are also enriched, which can be simultaneous with antigen-specific enrichment.

In various embodiments, the target peptide antigens are tumor or cancer associated antigens, including tumor-derived, tumor-specific antigens, and neoantigens. T cells specific for tumor associated antigens are often very rare, and in many cases undetectable, in the peripheral blood of healthy individuals. This is often a distinction observed between viral-specific and tumor antigen specific T cells.

In some embodiments, the target peptide antigens include at least one that is associated with or derived from a pathogen, such as a viral, bacterial, fungal, or parasitic pathogen. For example, at least one peptide antigen may be associated with HIV, hepatitis (e.g., B, C, or D) CMV, Epstein-Barr virus (EBV), influenza, herpes virus (e.g., HSV 1 or 2, or varicella zoster), and Adenovirus. CMV, for example, is the most common viral pathogen found in organ transplant patients and is a major cause of morbidity and mortality in patients undergoing bone marrow or peripheral blood stem cell transplants. Viral activation is known to be implicated in cancer biology.

In still other embodiments, the cell composition comprises T cells specific for tumor associated antigens, with pathogen-associated T cells provided as bystander cells. Specifically, by enriching for CD8+ T cells based on selection of both HLA-peptide and anti-CD28, bystander cells will be enriched, and expanded, particularly when using a T cell growth factor cocktail that can drive some non-specific expansion of these cells without antigen-specific activation. In these embodiments, while a large portion of the composition are T cells specific for the target peptides (e.g., from 5% to 75%), remaining T cells (from about 0.25% to about 25%) provide some reconstitution of the immune system for common pathogens, which is particularly beneficial after transplant or beneficial in cancers with viral etiology.

Some embodiments employ T cell growth factors during expansion, which affect proliferation and/or differentiation of T cells. Particularly useful cytokines include MIP-1β, IL-1β, IL-2, IL-4, IL-6, IL-7, IL-10, IL-12, IL-15, IL-21, IFN-γ. In these or other embodiments, the cells are expanded in culture in the presence of one, two, or three cytokines selected from MIP-1β, IL-1β, and IL-6. In some embodiments, the cytokines further comprise IL-10. Cells can be expanded in culture from 1 to 4 weeks, such as from about 10 to about 21 days.

In other aspects, the invention provides methods for manufacturing the cell compositions, including by enrichment and expansion with aAPCs as described herein. Specifically, after depletion of CD4+ cells from source lymphocytes (e.g., from a healthy donor), antigen-specific CD8+ T cells are enriched for T cells specific for the target peptide antigens, as well as CD28+ cells in some embodiments. Target cells can be enriched using nanoparticle or microparticle aAPCs, such as paramagnetic particles that activate T cells ex vivo by magnetic field induced clustering of cell surface receptors. Other materials, including latex or other polymeric-based particles can also be used to cluster cell surface receptors (without magnetic-induced clustering). Enriched T cells can then be rapidly expanded ex vivo, including with the use of reconstituted T cell growth factors (e.g., comprising factors selected from MIP-1β, IL-1β, IL-2, IL-4, IL-6, IL-7, IL-10, IL-12, IL-15, IL-21, IFN-γ). In some embodiments, the cells are expanded in culture in the presence of one, two, or three cytokines selected from MIP-1β, IL-1β, and IL-6, and optionally IL-10. In some embodiments, the growth factors comprise or consist essentially of IL-2, IL-4, IL-6, INF-γ, and IL-1β.

In other aspects, the invention provides methods for adoptive cell therapy, including methods for treating a patient with cancer, and/or patients that have undergone allogeneic stem cell transplantation, with or without lympho-deleting therapy, cyto-reductive therapy, immunomodulatory therapy (prior to administration of the cell therapy). The cell therapy may be further provided with or without cytokine support post treatment. In some embodiments, the patient has a hematological cancer, which in some embodiments has relapsed after allogeneic stem cell transplantation. In some embodiments, the patient has acute myelogenous leukemia (AML) or myelodysplastic syndrome. For example, in some embodiments, the cell composition comprises T cells specific for WT1, PRAME, Survivin, and Cyclin A peptide antigens. However, in other embodiments, the cancers include various types of solid tumors, including carcinomas, sarcomas, and lymphomas. Exemplary target peptide antigens are described herein.

In some embodiments, the patient has an infectious disease or is at risk for an infectious disease. For example, patients that have undergone HSCT are at particular risk for infectious disease, given the immunocompromised state. Infectious diseases that can be treated or prevented include those caused by bacteria, viruses, prions, fungi, parasites, helminths, etc. Such diseases include AIDS, hepatitis B/C, CMV infection, Epstein-Barr virus (EBV) infection, influenza, herpes virus infection (including shingles), and adenovirus infection.

Other aspects and embodiments will be apparent from the following detailed description.

### DESCRIPTION OF THE FIGURES

FIG. 1 shows that MART-1 specific T cells enriched and expanded ex vivo from donor lymphocytes show a polyfunctional phenotype, including intracellular staining for IL-2 (proliferation and memory), IFN-γ (activating other T cells, memory, upregulation of MHC), TNF-α (pro-inflammatory), and CD107A (granzyme release, cytotoxic activity). The majority of T cells show at least three functional phenotypes.

FIG. 2 shows that MART-1 and AML specific T cells enriched and expanded ex vivo from donor lymphocytes using paramagnetic aAPCs are predominately central memory (T_{cm}) and effector memory (Tₑₘ) phenotype.

FIG. 3A and FIG. 3B show that antigen-specific T cells can be enriched and expanded in batch. FIG. 3A and FIG. 3B also show batch enrichment and expansion of T cells specific for Prame₁₀₀ RHAMM, WT1, and Survivin antigenic peptides.

FIG. 4A and FIG. 4B show that the composition with individual stimulation and expansion has consistent levels of AML antigen-specific T cells. Individual stimulation and expansion process consistently generates ˜15% antigen-specific T cells.

FIG. 5 shows that simultaneous stimulation/expansion process generates AML-specific T cell frequencies comparable to individual stimulation/expansion. The composition shown prepared by batch stimulation/expansion has ˜47% antigen-specific T cells.

FIG. 6 shows that the generated T cells demonstrate antigen-specific killing of AML tumor cells (THP-1 cell line). AML specific T cells are directed at 5 epitopes from WT-1, PRAME, and Survivin.

FIG. 7 shows that the cytokine cocktail used for ex vivo expansion impacts the number and phenotype of resulting cells. Reconstituted T cell growth factor (TF) includes IL-1β, IL-2, IL-4, IL-6, IL-21, IFN-γ, and MIP1β.

FIG. 8A and FIG. 8B show the presence of virus-specific bystander T cells on day 7 after MART-1-specific enrichment and expansion.

FIG. 9 shows the presence of virus-specific bystander T cells on day 14 after MART-1-specific enrichment and expansion.

FIG. 10 shows the presence of virus-specific bystander T cells on day 14 after AML-specific enrichment and expansion. These cells were largely of a memory phenotype.

FIG. 11 shows detection of CMV-specific bystander T cells during MART-1 specific enrichment and expansion process. The percent of virus-specific bystander cells remains constant through Day 14, while the number and percent of MART-1 specific T cells rises dramatically.

FIG. 12A, FIG. 12B, FIG. 12C and FIG. 12D show detection of virus specific bystander cells on Day 14 after MART-1-specific enrichment and expansion using a recombinant T cell growth factor cocktail (IL-1β, IL-2, IL-4, IL-6, IL-21, IFN-γ, and MIP1-β), which improves expansion of these bystander cells.

FIG. 13 has two panels (FIG. 13A and FIG. 13B) showing the specificity and phenotype of Mart-1 specific T cells generated by the enrichment and expansion process using a recombinant T cell growth factor cocktail (IL-2, IL-4, IL-6, IFN-γ, and IL1-β). The Mart-1 specific T cells (FIG. 13A, right panel) constituted about 35% of the culture, and showed a central memory (˜89%) and effector memory (˜9%) phenotype. The total culture showed a phenotype of ˜66% central memory and ˜32% effector memory.

### DETAILED DESCRIPTION OF THE INVENTION

In various aspects and embodiments, the invention provides an isolated cell composition suitable for adoptive immunotherapy, as well as methods of manufacture for the cell compositions and methods of treatment with the cell compositions. The composition comprises, in a pharmaceutically acceptable carrier, at least about 10⁶ CD8+ T cells specific for target peptide antigen(s). In various embodiments, at least about 20% of T cells in the composition exhibit a central or effector memory phenotype, providing for a robust and durable adoptive therapy. The cell composition does not comprise T cells expressing a chimeric antigen receptor or a recombinant TCR, and therefore, in various embodiments, provides an alternative to these technologies that often produce more exhausted T cell phenotypes and less durable responses.

As used herein, the term “target peptide antigen(s)” or “target antigens” refers to peptide antigens employed ex vivo to enrich and/or expand the desired CD8+ cell population, for example in connection with artificial Antigen Presenting Cell (aAPC) or professional Antigen Presenting Cell (pAPC) platforms (e.g., dendritic cells). The aAPCs or pAPCs are employed to activate and expand CTLs from donor or patient lymphocytes. In some embodiments, the target peptide antigens are peptide epitopes loaded onto aAPCs for ex vivo enrichment and expansion of specific CD8+ T cells. Thus, the term “specific for the target peptide antigen” means that the T cell is antigen experienced with the target antigen.

In various embodiments, the cell composition comprises at least about 10⁷ CD8+ T cells specific for the target peptide antigens, or at least about 10⁸, at least about 10⁹, or at least about 10¹⁰ CD8+ T cells specific for the target peptide antigens, to provide robust destruction of target cells. In some embodiments, the cell composition contains from 1×10⁷ to 1×10⁹ CD8+ T cells specific for the target antigens, or in some embodiments from 5×10⁷ to 5×10⁸ CD8+ T cells specific for the target antigens. For example, the composition can comprise from about 5×10⁵ to about 5×10⁶ cells per ml, in a volume of from 50 to 200 ml. In certain embodiments, the volume of the composition is <100 ml (e.g., from 50 to 100 ml). The cells of the composition in various embodiments are at least 70% viable, and provided in a sterile medium, which may be a cryoprotectant medium (e.g., 10% DMSO).

The cells of the composition, which are predominately CD8+ cytotoxic lymphocytes (CTLs), are also substantially of a central or effector memory phenotype. CTLs generally include the following phenotypic populations: naive, T memory stem cell (T_{scm}), central memory, effector memory, and terminally differentiated memory cells. In accordance with embodiments of the invention, T cells specific for the target antigens are substantially composed of central memory and effector memory phenotypes. In some embodiments, T cells specific for the target antigens further comprise T memory stem cells (T_{scm}). The cell composition thereby provides a durable response, including in vivo persistence of antigen-specific T cells for at least about 1 month, or at least about 3 months, or at least about 6 months, or at least about 12 months, or at least about 18 months, or at least about two years in some embodiments.

A naive T cell has differentiated in bone marrow, and successfully undergone the positive and negative processes of central selection in the thymus. A naive T cell is considered mature and, unlike activated or memory T cells, has not encountered its cognate antigen. Naive T cells can be characterized by the surface expression of L-selectin (CD62L) and the absence of activation markers. In the naive state, T cells are generally quiescent and non-dividing. In accordance with this disclosure, naive T cells are defined as CD62L+ and CD45RA+.

Memory T cells include T memory stem cells (T_{scm}), central memory and effector memory T cells. Memory T cells have previously responded to their cognate antigen. At a second encounter with the cognate antigen, memory T cells can reproduce to mount a faster and stronger immune response. Memory T cells include at least effector and central memory subtypes. Memory T cell subtypes are long-lived and can quickly expand to large numbers of effector T cells upon re-exposure to their cognate antigen.

T memory stem cells (T_{scm}) are defined herein as CD45RA+ and as having at least two markers (or in some embodiments at least three or all four markers) selected from CXCR3+, CD95+, CD11a+, and CD58+. This memory subpopulation has the stem cell-like capacity for self-renewal, as well as the multipotent capacity to reconstitute the memory and effector T cell subpopulations. T_{scm} cells can represent a small fraction of circulating T lymphocytes (e.g., >5%), and have the ability to proliferate rapidly and release inflammatory cytokines in response to antigen re-exposure. Accordingly, T_{scm} cells are a subset of the memory T cell subpopulation. The T_{scm} cell phenotypes can be created and/or controlled using, as disclosed herein, an enrichment and expansion process with paramagnetic artificial Antigen Presenting Cells (aAPCs) and a recombinant T cell growth factor cocktail.

In accordance with this disclosure, central memory T cells (T_{cm} cells) are defined as CD62L+ and CD45RA−. This memory subpopulation is commonly found in the lymph nodes and in the peripheral circulation. Effector memory T cells (Tₑₘ cells) are defined as CD62L− and CD45RA−. These memory T cells lack lymph node-homing receptors and are thus found in the peripheral circulation and tissues. TEMRA stands for terminally differentiated effector memory cells re-expressing CD45RA. These cells do not have the capacity to divide, and are CD62L− and CD45RA+.

T_{cm} cells display a capacity for self-renewal, and in accordance with embodiments of the invention, are important for obtaining a long-lived effect. Tₑₘ cells also have some capacity for self-renewal, and strongly express genes essential to the cytotoxic function. Tₑₘᵣₐ cells also provide robust cytotoxic function, but do not display a capacity for self-renewal.

The compositions in various embodiments comprise CTLs that are substantially composed of T_{scm}, T_{cm} and Tₑₘ cells to balance duration of the effect versus potent destruction of the malignancy or other target cells.

In various embodiments, the T cells in the composition are at least about 30% central and effector memory cells, or at least about 40% central or effector memory cells, or at least about 50% central or effector memory T cells, or in some embodiments are at least about 70% central or effector memory cells, or at least about 80% central or effector memory T cells. In some embodiments, the memory cells are about 10:90 to about 90:10 central to effector memory cells. In some embodiments, the T cells in the composition are from about 25:75 to about 75:25 central to effector memory cells. In some embodiments, the memory T cells are from about 40:60 to about 60:40 central to effector memory T cells. The cell composition comprises less than about 20% terminally differentiated memory T cells (e.g., Tₑₘᵣₐ cells), or less than about 10% or less than about 5% or less than about 4% terminally differentiated memory T cells in some embodiments. In various embodiments, the CD8+ T cells contain no more than about 20% naive cells, or in some embodiments, no more than about 15% naive cells, or no more than about 10% naive cells, or no more than about 5% naive cells, or no more than about 4% naive cells, or no more than about 3% naive cells, or no more than about 2% naive cells, or no more than about 1.5%, or no more than about 1% naive cells. In various embodiments, the CD8+ T cells contain from about 5% to about 25% T_{scm} cells, or in some embodiments, from about 5% to about 20% T_{scm} cells, or from about 5% to about 15% T_{scm} cells.

In various embodiments, the T cells specific for the target antigens are at least about 30% central and effector memory cells, or at least about 40% central or effector memory cells, or at least about 50% central or effector memory T cells, or in some embodiments are at least about 70% central or effector memory cells, or at least about 80% central or effector memory T cells. In some embodiments, these memory cells are about 10:90 to about 90:10 central to effector memory cells. In some embodiments, these T cells are from about 25:75 to about 75:25 central to effector memory cells. In some embodiments, the memory T cells are from about 40:60 to about 60:40 central to effector memory T cells. The T cells specific for the target antigen(s) are less than about 20% terminally differentiated memory T cells (e.g., TEMRA cells), or less than about 10% or less than about 5% or less than about 4% terminally differentiated memory T cells. In various embodiments, the T cells specific for target antigens contain no more than about 20% naive cells, or in some embodiments, no more than about 15% naive cells, or no more than about 10% naive cells, or no more than about 5% naive cells, or no more than about 2%, or 1.5%, or 1% naive cells. In various embodiments, the T cells specific for target antigens contain from about 5% to about 25% T_{scm} cells, or in some embodiments, from about 5% to about 20% T_{scm} cells, or from about 5% to about 15% T_{scm} cells. This phenotype can be created by the enrichment and expansion process with paramagnetic artificial Antigen Presenting Cells (aAPCs).

In various embodiments, the cell composition is at least 90% T cells, or at least 95% T cells, or at least 98%, or at least 99% T cells. For purposes of this disclosure, T cells are characterized by CD3+ cells. The T cells are generally CD8+. For example, the isolated cell composition may be characterized by having less than about 10%, or less than about 5% CD4+ T cells, or in some embodiments, less than about 2%, less than about 1.5%, or less than about 1% CD4+ T cells. When expanding CD8+ T cells ex vivo, CD4+ cells have a tendency to overgrow the CD8+ cells and compete for growth signals, and are not necessary for a robust and durable response.

It has been described that the presence of polyfunctional CD4+ and CD8+ T cells correlates with response to cancer vaccine therapy with peptide neoantigens. Ott P A, et al., An immunogenic personal neoantigen vaccine for patients with melanoma, Nature 547(7662):217-221 (2017). CD4+ and CD8+ T cells are further described as being important for mediating tumor cell destruction. See, Tran E, Cancer immunotherapy based on mutation-specific CD4+ T cells in a patient with epithelial cancer. Science 344, 641-645 (2014); Sahin U, et al., Personalized RNA mutanome vaccines mobilize poly-specific therapeutic immunity against cancer, Nature 547(7662):222-226 (2017). With respect to this disclosure, it is believed that adoptive cell compositions need only provide substantial numbers of antigen-specific CD8+ T cells for a robust and durable response, and particularly where the antigen-specific CD8+ T cells are provided in sufficient numbers and are substantially of the central and effector memory phenotype. In various embodiments, the antigen-specific CD8+ T cells further comprise T memory stem cells.

In various embodiments, the cell composition is substantially CD28+.

In various embodiments, the antigen-specific T cells display a polyfunctional phenotype upon activation. For example, upon activation the T cells are positive for two or more of: intracellular staining for IL-2, which is a marker for proliferation and memory; IFN-γ production, which activates other T cells, and induces memory and upregulation of WIC); production of TNF-α, a pro-inflammatory marker; and CD107A, which is a marker for granzyme release and cytotoxic activity. In various embodiments, at least 30%, at least 40%, at least 50%, at least 60%, at least 70%, or at least 80% of the antigen-specific T cells display at least three of these markers. In various embodiments, at least 30%, at least 40%, at least 50%, at least 60%, at least 70%, or at least 80% of the antigen-specific T cells display all four of these markers. In some embodiments, polyfunctionality is assessed or quantified using target killing assays, which assess the ability of CD8+ cytotoxic T cells to lyse target cells presenting the peptide antigen in complex with MHC.

Cell compositions in accordance with various embodiments can be prepared by enrichment of CD8+ cells that are specific for the target antigen(s) (e.g., tumor associated antigens or viral-associated antigens). This cell population, even when predominately naive cells in the source lymphocytes, can be rapidly expanded in culture to arrive at the cell compositions described herein. CD4+ cells can be depleted (pre- or post-antigen-specific enrichment) from the lymphocytes using CD4+ cell depletion microbeads. Antigen specific enrichment of CD8+ cells can take place using paramagnetic beads to positively select cell populations, and which can have the added advantage of activating naive cells due to potent magnetic clustering of T cell surface receptors. For example, paramagnetic beads or nanoparticles may contain monomeric or multimeric (e.g., dimeric) HLA ligands presenting peptide antigens, along with a co-stimulation signal in some embodiments, such as an agonist for CD28 (e.g., an antibody agonist of CD28). Exemplary methods according to these embodiments are described in WO 2016/044530 and PCT/US2017/22663, which are hereby incorporated by reference in its entirety.

In some embodiments, CD28+ cells are also enriched, which can be simultaneous with antigen-specific enrichment. CD28 is expressed on T cells, and is a co-stimulatory signal required for T cell activation and survival. CD28 is the only B7 receptor constitutively expressed on naive T cells. Association of the TCR of a naive T cell with MHC-antigen complex without CD28 co-stimulation can result in a T cell that is anergic. In some embodiments, CD28+ cells are not enriched, but a CD28 agonist is added in soluble form during the enrichment process, or added as conjugated to non-paramagnetic beads. In some embodiments, CD28 (in conjugated or non-conjugated form) is added to the cells after antigen-specific enrichment, in order to activate cells for the expansion phase.

In various embodiments, the T cells specific for target antigens (e.g., by virtue of the peptides displayed by the aAPCs or pAPCs) are specific for from 1 to about 100 target antigens, or from 1 to about 75 target antigens, or from 1 to about 50 target antigens, or from 1 to about 25 target antigens, or from 1 to about 20 target antigens, or from 1 to about 15 target antigens, or from 1 to 10 target antigens, or from 1 to 5 target antigens. In various embodiments, there are at least 3, or at least 4, or at least 5 target antigens. The distinct target antigens can include overlapping peptide epitopes in some embodiments. T cells specific for these peptide antigens can be enriched and expanded in batch, allowing for rapid, parallel production of cell compositions. In some embodiments, the composition contains T cells specific for from 5 to 15 or from 5 to 10 peptide antigens. T cell specificity toward a target peptide antigen in the composition is defined by MHC multimer staining (e.g., dimer or tetramer staining) as is well known in the art.

For example, a cocktail of nano-aAPCs, each aAPC presenting a different, distinct target antigen, can be used to enrich T cells against multiple antigens simultaneously. For example, T cells specific for from 2 to 10 antigens can be enriched simultaneously from the lymphocyte source. In this embodiment, a number of different nano-aAPC batches, each bearing a different MHC-peptide, would be combined and used to simultaneously enrich T cells against each of the antigens of interest. The resulting T cell pool would be activated against each of these antigens, and expanded together in culture. These antigens could be related to a single therapeutic intervention; for example, multiple antigens present on a single tumor or malignant cell.

The target peptide antigens are generally suitable for presentation by an HLA-A, B, or C molecular complex, and in some embodiments an HLA-A2 molecular complex.

In various embodiments, the target peptide antigens are tumor or cancer associated antigens, including tumor-derived or tumor-specific antigens. T cells specific for tumor associated antigens are often very rare, and in many cases undetectable, in the peripheral blood of healthy individuals. Further, the cells are often of a naive phenotype, particularly when using donor T lymphocytes. See, Quintarelli et al., Cytotoxic T lymphocytes directed to the preferentially expressed antigens of melanoma (PRAME) target chronic myeloid leukemia. Blood 2008; 112: 1876-1885. This is often a distinction observed between viral-specific and tumor antigen specific T cells.

“Tumor-associated antigens” or “cancer specific antigens” include unique tumor or cancer antigens expressed exclusively by the tumor or malignant cells from which they are derived, shared tumor antigens expressed in many tumors but not in normal adult tissues (oncofetal antigens), and tissue-specific antigens expressed also by the normal tissue from which the tumor arose. Tumor associated antigens can be, for example, embryonic antigens, antigens with abnormal post-translational modifications, differentiation antigens, products of mutated oncogenes or tumor suppressors, fusion proteins, or oncoviral proteins.

In some embodiments, the target peptide antigens include one or more associated with or derived from hematological cancer, such as leukemia, lymphoma, or myeloma. For example, the hematological malignancy may be acute myeloid leukemia, chronic myelogenous leukemia, childhood acute leukemia, non-Hodgkin's lymphomas, acute lymphocytic leukemia, chronic lymphocytic leukemia, myelodysplastic syndrome, malignant cutaneous T-cells, mycosis fungoids, non-MF cutaneous T-cell lymphoma, lymphomatoid papulosis, and T-cell rich cutaneous lymphoid hyperplasia. In other embodiments, the target peptide antigens include one or more associated with or derived from a solid tumor, including melanoma, colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, neuroblastoma, and glioma.

A variety of tumor-associated antigens are known in the art. Oncofetal and embryonic antigens include carcinoembryonic antigen and alpha-fetoprotein (usually only highly expressed in developing embryos but frequently highly expressed by tumors of the liver and colon, respectively), MAGE-1 and MAGE-3 (expressed in melanoma, breast cancer, and glioma), placental alkaline phosphatase sialyl-Lewis X (expressed in adenocarcinoma), CA-125 and CA-19 (expressed in gastrointestinal, hepatic, and gynecological tumors), TAG-72 (expressed in colorectal tumors), epithelial glycoprotein 2 (expressed in many carcinomas), pancreatic oncofetal antigen, 5T4 (expressed in gastriccarcinoma), alphafetoprotein receptor (expressed in multiple tumor types, particularly mammary tumors), and M2A (expressed in germ cell neoplasia).

Tumor-associated differentiation antigens include tyrosinase (expressed in melanoma) and particular surface immunoglobulins (expressed in lymphomas).

Mutated oncogene or tumor-suppressor gene products include Ras and p53, both of which are expressed in many tumor types, Her-2/neu (expressed in breast and gynecological cancers), EGF-R, estrogen receptor, progesterone receptor, retinoblastoma gene product, myc (associated with lung cancer), ras, p53, nonmutant associated with breast tumors, MAGE-1, and MAGE-3 (associated with melanoma, lung, and other cancers). Fusion proteins include BCR-ABL, which is expressed in chromic myeloid leukemia. Oncoviral proteins include HPV type 16, E6, and E7, which are found in cervical carcinoma.

Tissue-specific antigens include melanotransferrin and MUC1 (expressed in pancreatic and breast cancers); CD10 (previously known as common acute lymphoblastic leukemia antigen, or CALLA) or surface immunoglobulin (expressed in B cell leukemias and lymphomas); the α chain of the IL-2 receptor, T cell receptor, CD45R, CD4+/CD8+(expressed in T cell leukemias and lymphomas); prostate specific antigen and prostatic acid-phosphatase (expressed in prostate carcinoma); GP 100, MelanA/Mart-1, tyrosinase, gp75/brown, BAGE, and S-100 (expressed in melanoma); cytokeratins (expressed in various carcinomas); and CD19, CD20, and CD37 (expressed in lymphoma).

Tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas).

For example, in some embodiments, one or more target antigens are associated with bladder cancer, such as one or more of NY-ESO-1, MAGE-A10, and MUC-1 antigens. In some embodiments, one or more target antigens are associated with brain cancer, and may include one or more of NY-ESO-1, Survivin, and CMV antigens. In some embodiments, one or more target antigens are associated with breast cancer, and may include one or more of MUC-1, Surivin, WT-1, HER-2, and CEA antigens. In some embodiments, one or more target antigens are associated with cervical cancer, and may include HPV antigen. In some embodiments, one or more target antigens are associated with colorectal cancer, and may include one or more of NY-ESO-1, Survivin, WT-1, MUC-1, and CEA antigens. In some embodiments, one or more target antigens are associated with esophageal cancer, and may include NY-ESO-1 antigen. In some embodiments, one or more target antigens may be associated with head and neck cancer, and may include HPV antigen. In some embodiments, the target antigen is associated with kidney or liver cancer, and may include NY-ESO-1 antigen. In some embodiments, the target antigen is associated with lung cancer, and may include one or more of NY-ESO-1, Survivin, WT-1, MAGE-A10, and MUC-1 antigens. In some embodiments, one or more target antigens is associated with melanoma, and may include one or more of NY-ESO-1, Survivin, MAGE-A10, MART-1, and GP-100. In some embodiments, one or more peptide antigens are associated with ovarian cancer, and may include one or more of NY-ESO-1, WT-1, and Mesothelin antigen. In some embodiments, one or more target antigens are associated with prostate cancer, and may include one or more of Survivin, hTERT, PSA, PAP, and PSMA antigens. In some embodiments, the target antigen is associated with a sarcoma, and may include NY-ESO-1 antigen. In some embodiments, one or more target antigens are associated with lymphoma, and may include EBV antigen. In some embodiments, one or more target antigens are associated with multiple myeloma, and may include one or more of NY-ESO-1, WT-1, and SOX2 antigens.

In some embodiments, one or more target antigens are associated with acute myelogenous leukemia or myelodysplastic syndrome, and may include one or more of (including 1, 2, 3, 4, or 5 of) Survivin, WT-1, PRAME, RHAMM, PR3, and Cyclin A1 antigens. In some embodiments, the target antigens include at least 1, 2, 3, 4, 5, 6, 7, 8, 9, 10 or all target antigens from Table 1 below.

TABLE 1
Exemplary AML target peptide antigens

| | Peptide | | |
|---|---|---|---|
| Antigen | name/position | Sequence | SEQ ID NO: |
| WT-1 | 126-134 | RMFPNAPYL | SEQ ID NO: 1 |
| | 235-243 | CMTWNQMNL | SEQ ID NO: 2 |
| | 37-45 | VLDFAPPGA | SEQ ID NO: 3 |
| | 187-195 | SLGEQQYSV | SEQ ID NO: 4 |
| Prame | P100 | VLDGLDVLL | SEQ ID NO: 5 |
| | P435 | NLTHVLYPV | SEQ ID NO: 6 |
| | P142 | SLYSFPEPEA | SEQ ID NO: 7 |
| | P300 | ALYVDSLFFL | SEQ ID NO: 8 |
| | P425 | SLLQHLIGL | SEQ ID NO: 9 |
| Survivin | ELT 95-104 | ELTLGEFLKL | SEQ ID NO: 10 |
| | LDR 104-113 | LDRERAKNKI | SEQ ID NO: 11 |
| Cyclin A1 | 227-235 | FLDRFLSCM | SEQ ID NO: 12 |
| | 341-351 | SLIAAAAFCLA | SEQ ID NO: 13 |

In some embodiments, one or more target peptide antigens are neoantigens. For example, in some embodiments, neoantigens specific to the patient are identified, and synthesized for loading aAPCs. In some embodiments, between three and ten neoantigens are identified through genetic analysis of the patient's malignancy (e.g., by nucleic acid sequencing of malignant cells), followed by predictive bioinformatics. In some embodiments, the antigens are natural, non-mutated, cancer antigens, of which many are known.

In various embodiments, at least one of the target peptide antigens is recognized by a low frequency precursor T cell. In accordance with these embodiments, the invention enables rapid activation and expansion of these cells for adoptive therapy.

In some embodiments, the target peptide antigens include at least one that is associated with or derived from a pathogen, such as a viral, bacterial, fungal, or parasitic pathogen. For example, at least one peptide antigen may be associated with HIV, hepatitis (e.g., A, B, C, or D) CMV, Epstein-Barr virus (EBV), influenza, herpes virus (e.g., HSV 1 or 2, or varicella zoster), and Adenovirus. CMV, for example, is the most common viral pathogen found in organ transplant patients and is a major cause of morbidity and mortality in patients undergoing bone marrow or peripheral blood stem cell transplants. This is due to the immunocompromised status of these patients, which permits reactivation of latent virus in seropositive patients or opportunistic infection in seronegative individuals. In these embodiments, the patient may receive adoptive immunotherapy comprising T cells specific for pathogen antigens. The method can entail generation of virus-specific CTL derived from the patient or from an appropriate donor before initiation of the transplant procedure.

In some embodiments, at least one target antigen is a pathogen-associated antigen, including antigens associated with protozoa, bacteria, fungi (both unicellular and multicellular), viruses, prions, intracellular parasites, helminths, and other infectious agents.

Bacterial antigens include antigens of gram-positive cocci, gram positive bacilli, gram-negative bacteria, anaerobic bacteria, such as organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaceae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma.

Antigens of protozoan infectious agents include antigens of malarial plasmodia, Leishmania species, Trypanosoma species and Schistosoma species.

Fungal antigens include antigens of Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia.

Viral peptide antigens include, but are not limited to, those of adenovirus, herpes simplex virus, papilloma virus, respiratory syncytial virus, poxviruses, HIV, influenza viruses, EBV, hepatitis, and CMV. Particularly useful viral peptide antigens include HIV proteins such as HIV gag proteins (including, but not limited to, membrane anchoring (MA) protein, core capsid (CA) protein and nucleocapsid (NC) protein), HIV polymerase, influenza virus matrix (M1) protein and influenza virus nucleocapsid (NP) protein, hepatitis B surface antigen (HBsAg), hepatitis B core protein (HBcAg), hepatitis e protein (HBeAg), hepatitis B DNA polymerase, hepatitis C antigens, and the like.

In some embodiments, the target peptide antigens include one or more tumor associated antigens, and one or more virus-associated antigens (such as CMV, EBV, influenza, or Adenovirus), to provide an antitumor response while protecting against common pathogens that complicate recovery after HSCT.

Patients that have undergone HSCT are at particular risk for infectious disease, given the immunocompromised state. The immunocompromised status of these patients permits reactivation of latent virus in seropositive patients or opportunistic infection in seronegative individuals. For example, Post-transplant lymphoproliferative disease (PTLD) occurs in a significant fraction of transplant patients and results from Epstein-Barr virus (EBV) infection. EBV infection is believed to be present in approximately 90% of the adult population in the United States. Active viral replication and infection is kept in check by the immune system, but, as in cases of CMV, individuals immunocompromised by transplantation therapies lose the controlling T cell populations, which permits viral reactivation. This represents a serious impediment to transplant protocols. EBV may also be involved in tumor promotion in a variety of hematological and non-hematological cancers.

In still other embodiments, the cell composition comprises T cells specific for tumor associated antigens, with pathogen-associated T cells provided as bystander cells. Specifically, by enriching for CD8+ T cells based on selection with both HLA-peptide complexes and anti-CD28, bystander cells will be enriched, and expanded, particularly when using a T cell growth factor cocktail that can drive some non-specific expansion of these cells without antigen-specific activation. In these embodiments, while a large portion of the composition are T cells specific for the target peptides (e.g., from 5% to 75%, or from 10 to 50%), the remaining T cells provide some reconstitution of the immune system against common pathogens, which is particularly beneficial after transplant. For example, the composition may comprise T cells specific for CMV, EBV, influenza, and Adenovirus. In each case, pathogen-specific T cells may be present at from 0.1% to about 4% of the composition.

In various embodiments the invention involves compositions prepared by enrichment and expansion of antigen-specific CD8+ T cells. Precursor T cells can be obtained from the patient or from a suitable HLA-matched donor. Source T cells can be either fresh or frozen samples. Precursor T cells can be obtained from a number of sources that comprise WBCs, including peripheral blood mononuclear cells (PBMC), bone marrow, lymph node tissue, spleen tissue, buffy coat fraction, and tumors. In some embodiments, precursor T cells are obtained from a unit of blood collected from a subject using any number of techniques known to one or skill in the art. For example, precursor T cells from the circulating blood of an individual can be obtained by apheresis or leukapheresis. The apheresis product typically contains lymphocytes, including T cells and precursor T cells, monocytes, granulocytes, B cells, other nucleated white blood cells, red blood cells, and platelets. Leukapheresis is a laboratory procedure in which white blood cells are separated from a sample of blood.

Cells collected by apheresis can be washed to remove the plasma fraction and to place the cells in an appropriate buffer or media for subsequent processing steps. Washing steps can be accomplished by methods known to those in the art, such as by using a semi-automated “flow-through” centrifuge. After washing, the cells may be resuspended in a variety of biocompatible buffers, such as, for example, Ca-free, Mg-free PBS. Alternatively, the undesirable components of the apheresis sample can be removed and the cells directly re-suspended in a culture medium.

If desired, precursor T cells can be isolated from peripheral blood lymphocytes by lysing the red blood cells and depleting the monocytes, for example, by centrifugation through a PERCOLL™ gradient.

In certain embodiments, leukocytes are collected by leukapheresis, and may be subsequently enriched for CD8+ T cells, for example, by depleting the sample of CD4+ cells and/or positively enriching for CD8+ cells. In some embodiments, other cell types are depleted, such as NK cells. The CD8-enriched cells may then be further enriched for antigen-specific T cells.

In various embodiments, the sample comprising the immune cells (e.g., CD8+ T cells) is contacted with an artificial Antigen Presenting Cell (aAPC) having magnetic properties. Paramagnetic materials have a small, positive susceptibility to magnetic fields. These materials are attracted by a magnetic field and the material does not retain the magnetic properties when the external field is removed. Exemplary paramagnetic materials include, without limitation, magnesium, molybdenum, lithium, tantalum, and iron oxide. Paramagnetic beads suitable for magnetic enrichment are commercially available (DYNABEADS™, MACS MICROBEADS™, Miltenyi Biotec). In some embodiments, the aAPC particle is an iron dextran bead (e.g., dextran-coated iron-oxide bead).

Antigen presenting complexes comprise an antigen binding cleft, and are generally MHC class I, which can be linked or tethered to provide dimeric or multimeric MHC. In some embodiments, the MHC are monomeric, but their close association on the nano-particle is sufficient for avidity and activation. In some embodiments, the MHC are dimeric. Dimeric MHC class I ligands can be constructed by fusion to immunoglobulin heavy chain sequences, which are then associated through one or more disulfide bonds (with or without associated light chains). MHC multimers can be created by direct tethering through peptide or chemical linkers, or can be multimeric via association with streptavidin through biotin moieties. In some embodiments, the antigen presenting complexes are MHC class I complexes involving fusions with immunoglobulin sequences.

MHC class I molecular complexes having immunoglobulin sequences are described in U.S. Pat. No. 6,268,411, which is hereby incorporated by reference in its entirety. These MHC class I molecular complexes may be formed in a conformationally intact fashion at the ends of immunoglobulin heavy chains. MHC class I molecular complexes to which antigenic peptides are bound can stably bind to antigen-specific lymphocyte receptors (e.g., T cell receptors). In various embodiments, the immunoglobulin heavy chain sequence is not full length, but comprises an Ig hinge region, and one or more of CH1, CH2, and/or CH3 domains. The Ig sequence may or may not comprise a variable region, but where variable region sequences are present, the variable region may be full or partial. The complex may further comprise immunoglobulin light chains. MHC class I ligands (e.g., HLA-Ig) lacking variable chain sequences (and lacking any light chain) may be employed with site-directed conjugation to particles, as described in WO 2016/105542, which is hereby incorporated by reference in its entirety.

Exemplary MHC class I molecular complexes comprise at least two fusion proteins. A first fusion protein comprises a first MHC class I a chain and a first immunoglobulin heavy chain (or portion thereof comprising the hinge region), and a second fusion protein comprises a second MHC class I a chain and a second immunoglobulin heavy chain (or portion thereof comprising the hinge region). The first and second immunoglobulin heavy chains associate to form the MHC class I molecular complex, which comprises two MHC class I peptide-binding clefts. The immunoglobulin heavy chain can be the heavy chain of an IgM, IgD, IgG1, IgG3, IgG2β, IgG2α, IgG4, IgE, or IgA. In some embodiments, an IgG heavy chain is used to form MHC class I molecular complexes. If multivalent MHC class I molecular complexes are desired, IgM or IgA heavy chains can be used to provide pentavalent or tetravalent molecules, respectively.

Exemplary class I molecules include HLA-A, HLA-B, HLA-C, HLA-E, and these may be employed individually or in any combination. In some embodiments, the antigen presenting complex is an HLA-A2 ligand. The term MHC as used herein, can be replaced by HLA in each instance.

Immunoglobulin sequences in some embodiments are humanized monoclonal antibody sequences.

The aAPCs may contain a “Signal 2”, such as an anti-CD28 ligand. Signal 2 is generally a T cell affecting molecule, that is, a molecule that has a biological effect on a precursor T cell or on an antigen-specific T cell. In certain embodiments, signal 2 is a T cell costimulatory molecule. T cell costimulatory molecules contribute to the activation of antigen-specific T cells. Such molecules include, but are not limited to, molecules that specifically bind to CD28 (including antibodies), CD80 (B7-1), CD86 (B7-2), B7-H3, 4-1BB, 4-1BBL, CD27, CD30, CD134 (OX-40L), B7h (B7RP-1), CD40, LIGHT, antibodies that specifically bind to HVEM, antibodies that specifically bind to CD40L, and antibodies that specifically bind to OX40. In some embodiments, the costimulatory molecule (signal 2) is an antibody (e.g., a monoclonal antibody) or portion thereof, such as F(ab′)2, Fab, scFv, or single chain antibody, or other antigen binding fragment. In some embodiments, the antibody is a humanized monoclonal antibody or portion thereof having antigen-binding activity, or is a fully human antibody or portion thereof having antigen-binding activity.

Combinations of co-stimulatory ligands that may be employed (on the same or separate nanoparticles) include anti-CD28/anti-CD27 and anti-CD28/anti-41BB. The ratios of these co-stimulatory ligands can be varied to effect expansion.

Exemplary signal 1 and signal 2 ligands are described in WO 2014/209868, which describe ligands having a free sulfhydryl (e.g., unpaired cysteine), such that the constant region may be coupled to nanoparticle supports having the appropriate chemical functionality.

Adhesion molecules useful for nano-aAPC can be used to mediate adhesion of the nano-aAPC to a T cell or to a T cell precursor. Useful adhesion molecules include, for example, ICAM-1 and LFA-3.

In some embodiments, signal 1 is provided by peptide-HLA-A2 complexes, and signal 2 is provided by B7.1-Ig or anti-CD28. An exemplary anti-CD28 monoclonal antibody is 9.3 mAb (Tan et al., J. Exp. Med. 1993 177:165), which may be humanized in certain embodiments and/or conjugated to the bead as a fully intact antibody or an antigen-binding fragment thereof.

Magnetic activation may take place for from 2 minutes to 5 hours, or from 5 minutes to 2 hours, followed by expansion in culture for at least 5 days, and up to 2 weeks or up to 3 weeks in some embodiments. In some embodiments, magnetic activation occurs for at least 2 minutes, but less than 30 minutes (e.g., about 5 or 10 minutes). Resulting CD8+ T cells may be phenotypically characterized to confirm the presence of T memory stem cells (T_{scm}), as well as high central and effector memory phenotype.

Some embodiments employ T cell growth factors during expansion, which affect proliferation and/or differentiation of T cells. Examples of T cell growth factors include cytokines (e.g., interleukins, interferons) and superantigens. If desired, cytokines can be present in molecular complexes comprising fusion proteins, or can be encapsulated by the aAPC, or provided in soluble form. Particularly useful cytokines include MIP-1β, IL-1β, IL-2, IL-4, IL-6, IL-7, IL-10, IL-12, IL-15, IL-21, IFN-γ, and CXCL10. In some embodiments, the growth factors include 3, 4, 5, or 6 from MIP-1β, IL-1β, IL-2, IL-4, IL-6, IL-7, IL-15, IL-21, and INF-γ. In these or other embodiments, the cells are expanded in culture in the presence of one, two, three cytokines selected from MIP-1β, IL-1β, and IL-6, and optionally IL-10. In some embodiments, the cells are not cultured in the presence of IL-7 and/or IL-21 and/or IL-15. Cells can be expanded in culture from 1 to 4 weeks, such as about 2 weeks (about 14 days), or about 3 weeks.

In some embodiments, the cells are expanded in culture in the presence of from 4 to 8 cytokines, to achieve a balance between T cell expansion (including antigen-specific T cell expansion), activation, and memory phenotype. In some embodiments, the cells are expanded in the presence of IL-4. In some embodiments, the cells are expanded in the presence of IL-4 and IL-6. In some embodiments, the cells are expanded in the presence of IL-4 and IL-1β. In some embodiments, the cells are expanded in the presence of IL-4, IL-6, and IL-1β. In some embodiments, the cells are expanded in the presence of IL-2, IL-4, and IL-6. In some embodiments, the cells are expanded in culture in the presence of IL-2, IL-4, IL-6, INF-γ, and IL-1β. In some embodiments, the cells are further expanded in the presence of IL-10.

In some embodiments, the growth factors consist, or consist essentially of, IL-2, IL-4, IL-6, INF-γ, and IL-1β, and optionally IL-10.

In some embodiments, IL-2 is present at the start of culture at 10 to 200 International Units (IU) per ml, such as from about 20 to about 100 IU/ml, or about 20 to about 60 IU/ml. In some embodiments, IL-2 is present at the start of culture at about 30 to about 50 IU/ml (e.g., about 40 IU/ml). IL-2 IU (86/500 NIBSC) can be determined using a proliferation assay (e.g., using CTLL-2 cell line), as described for example by Gearing and Bird (1987) in Lymphokines and Interferons, A Practical Approach. Clemens, M J et al. (eds): IRL Press. 295. In some embodiments, IL-2 is present at the start of culture at about 2 to about 25 ng/ml, such as from about 5 to about 15 ng/ml.

In these or independent embodiments, IL-4 is present at the start of culture at 0.2 to 25 International Units (IU) per ml, such as from about 0.5 to about 10 IU/ml, or from about 0.5 to about 5 IU/ml. In some embodiments, IL-4 is present at the start of culture at about 1 IU/ml. IL-4 IU (88/656 NIBSC) can be defined using a proliferation assay (e.g., using TF-1 cell line), as described for example, by Kitamura T. et al., (1991) IL-1 up-regulates the expression of cytokine receptors on a factor-dependent human hemopoietic cell line, TF-1. Int. Immunol. 3:571-577. In some embodiments, IL-4 is present at the start of culture at about 0.2 to about 2 ng/ml, such as from about 0.2 to about 1 ng/ml (e.g., about 0.5 ng/ml).

In these or independent embodiments, IL-6 may be present at the start of culture at 10 to 200 International Units (IU) per ml, such as from about 25 to about 100 IU/ml, such as from 25 to 75 IU/ml. In some embodiments, IL-6 is present at the start of culture at about 40 to about 60 IU/ml (e.g., about 50 IU/ml). IL-6 IU (89/548 NIBSC) can be defined using a proliferation assay (e.g., using B9 cell line), as described for example by Gaines-Das R E and Poole S. (1993) The international standard for interleukin-6. Evaluation in an international collaborative study. J. Immunol. Methods 160:147-153. In some embodiments, IL-6 is present at the start of culture at about 0.2 to about 10 ng/ml, such as from about 0.2 to about 5 ng/ml (e.g., about 0.5 to 2 ng/ml).

In these or independent embodiments, Interferon gamma (INF-γ) may be present at the start of culture at from 10 to 200 International Units (IU) per ml, such as from about 20 to about 100 IU/ml, such as from 20 to 60 IU/ml. In some embodiments, INF-γ is present at the start of culture at about 30 to about 50 IU/ml (e.g., about 40 IU/ml). INF-γ IU (87/586 NIBSC) can be defined using an antiviral assay (e.g., with Hela cells infected with EMC), as described for example in Meager A. (1987) in Lymphokines and interferons, a Practical Approach. Clemens, M J, et al. (eds): IRL Press. 129. In some embodiments, INF-γ is present at the start of culture at about 0.5 to about 20 ng/ml, such as from about 1 to about 10 ng/ml (e.g., from 1 to 5 ng/ml).

IL-1β may be present at the start of culture at 5 to 100 International Units (IU) per ml, such as from about 10 to about 50 IU/ml, such as from about 10 to about 30 IU/ml. In some embodiments, IL-1β is present at the start of culture at about 10 to about 20 IU/ml (e.g., about 15 IU/ml). IL-1β IU (86/680 NIBSC) can be defined using a proliferation assay (e.g., using D.10.G4.1 cells), as described for example by Poole, S. and Gaines-Das, R E (1991) The international standards for interleukin-1 alpha and interleukin-1 beta. Evaluation in an international collaborative study. J. Immunol. Methods 142:1-13. In some embodiments, IL-1β is present at the start of culture at about 0.2 to about 5 ng/ml, such as from about 0.2 to about 2 ng/ml, or from about 0.2 to about 1 ng/ml.

In various embodiments, the cells are cultured in the presence of a growth factor cocktail comprising or consisting of IL-2, IL-4, IL-6, INF-γ, and IL-1β. In some embodiments, the relative activity (defined by the respective IU) of IL-2 and INF-γ is about 0.5:1 to about 1:0.5 (e.g., about 1:1). In these or independent embodiments, the relative activity (defined by respective IU) of IL-2 and IL-6 is about 0.5:1 to 1:0.5. In these or independent embodiments, the relative activity of IL-1β with respect to IL-2, IL-6, and/or IFN-γ (defined by respective IUs) is from 1:4 to 1:2 (e.g., about 1:3). In these or independent embodiments, the relative activity of IL-4 with respect to IL-2, IL-6, and/or IFN-γ (defined by respective IUs) is from 1:30 to 1:60. In these or independent embodiments, the relative activity of IL-4 with respect to IL-1β (defined by respective IUs) is from about 1:5 to about 1:25, such as from about 1:10 to about 1:20.

In some embodiments, the specific activity of each growth factor (IL-2, IL-4, IL-6, INF-γ, and IL-1β) at the start of culture (in IUs) can be shown as a percentage when the total IUs of all the growth factors in the culture is considered as 100%. For example, in some embodiments, the percentage of each growth factor in the culture can be as follows:

20% to 40% IL-2 (e.g., 20 to 30% IL-2);

0.5% to 5% IL-4 (e.g., 1 to 3% IL-4);

25% to 50% IL-6 (e.g., 30 to 40% IL-6);

20% to 40% IFN-γ (e.g., 20 to 30% IFN-γ); and

5% to 20% IL-1β (e.g., 5 to 15% IL-1β).

The aAPC nanoparticles can be made of any material, and materials can be appropriately selected for the desired magnetic property, and may comprise, for example, metals such as iron, nickel, cobalt, or alloy of rare earth metal. Paramagnetic materials also include magnesium, molybdenum, lithium, tantalum, and iron oxide. Paramagnetic beads suitable for enrichment of materials (including cells) are commercially available, and include iron dextran beads, such as dextran-coated iron oxide beads. In aspects of the invention where magnetic properties are not required, nanoparticles can also be made of nonmetal or organic (e.g., polymeric) materials such as cellulose, ceramics, glass, nylon, polystyrene, rubber, plastic, or latex. In exemplary material for preparation of nanoparticles is poly(lactic-co-glycolic acid) (PLGA) or PLA and copolymers thereof, which may be employed in connection with these embodiments. Other materials including polymers and co-polymers that may be employed include those described in PCT/US2014/25889, which is hereby incorporated by reference in its entirety.

In various embodiments, the particle has a size (e.g., average diameter) within about 10 to about 500 nm, or within about 40 to about 400 nm, or within about 100 nm to 400 nm. For magnetic clustering, it is preferred that the nanoparticles have a size in the range of 10 to 250 nm, or 20 to 100 nm in some embodiments. Receptor-ligand interactions at the cell-nanoparticle interface are not well understood. However, nanoparticle binding and cellular activation are sensitive to membrane spatial organization, which is particularly important during T cell activation, and magnetic fields can be used to manipulate cluster-bound nanoparticles to enhance activation. For example, T cell activation induces a state of persistently enhanced nanoscale TCR clustering and nanoparticles are sensitive to this clustering in a way that larger particles are not.

Furthermore, nanoparticle interactions with TCR clusters can be exploited to enhance receptor triggering. T cell activation is mediated by aggregation of signaling proteins, with “signaling clusters” hundreds of nanometers across, initially forming at the periphery of the T cell-APC contact site and migrating inward. As described herein, an external magnetic field can be used to enrich antigen-specific T cells (including rare naive cells) and to drive aggregation of magnetic nano-aAPC bound to TCR, resulting in aggregation of TCR clusters and enhanced activation of naive T cells. Magnetic fields can exert appropriately strong forces on paramagnetic particles, but are otherwise biologically inert, making them a powerful tool to control particle behavior. T cells bound to paramagnetic nano-aAPC are activated in the presence of an externally applied magnetic field. Nano-aAPC are themselves magnetized, and attracted to both the field source and to nearby nanoparticles in the field, inducing bead and thus TCR aggregation to boost aAPC-mediated activation.

Activation chemistries can be used to allow the specific, stable attachment of molecules to the surface of nanoparticles. There are numerous methods that can be used to attach proteins to functional groups. For example, the common cross-linker glutaraldehyde can be used to attach protein amine groups to an aminated nanoparticle surface in a two-step process. The resultant linkage is hydrolytically stable. Other methods include use of cross-linkers containing n-hydrosuccinimido (NETS) esters which react with amines on proteins, cross-linkers containing active halogens that react with amine-, sulfhydryl-, or histidine-containing proteins, cross-linkers containing epoxides that react with amines or sulfhydryl groups, conjugation between maleimide groups and sulfhydryl groups, and the formation of protein aldehyde groups by periodate oxidation of pendant sugar moieties followed by reductive amination.

The ratio of particular ligands when used simultaneously on the same or different particles can be varied to increase the effectiveness of the nanoparticle in antigen or costimulatory ligand presentation. For example, nanoparticles can be coupled with HLA-A2-Ig and anti-CD28 (or other signal 2 ligands) at a variety of ratios, such as about 30:1, about 25:1, about 20:1, about 15:1, about 10:1, about 5:1, about 3:1, about 2:1, about 1:1, about 0.5:1, about 0.3:1; about 0.2:1, about 0.1:1, or about 0.03:1. In some embodiments, the ratio is from 2:1 to 1:2. The total amount of protein coupled to the supports may be, for example, about 250 mg/ml, about 200 mg/ml, about 150 mg/ml, about 100 mg/ml, or about 50 mg/ml of particles. Because effector functions such as cytokine release and growth may have differing requirements for Signal 1 versus Signal 2 than T cell activation and differentiation, these functions can be determined separately.

In certain embodiments, the aAPCs are paramagnetic particles in the range of 50 to 150 nm, with a PDI (size distribution) of less than 0.2, or in some embodiments less than 0.1. The aAPCs may have a surface charge of from 0 to −10 mV, such as from about −2 to −6 mV. aAPCs may have from 10 to 120 ligands per particle, such as from about 25 to about 100 ligands per particle, with ligands conjugated to the particle through a free cysteine introduced in the Fc region of the immunoglobulin sequences. The particles may contain about 1:1 ratio of HLA dimer:anti-CD28, which may be present on the same or different populations of particles. The nanoparticles provide potent expansion of cognate T cells, while exhibiting no stimulation of non-cognate TCRs, even with passive loading of peptide antigen. Particles are stable in lyophilized form for at least two or three years.

After enrichment and expansion, the antigen-specific T cell component of the sample will be at least about 5%, or at least about 10%, or at least about 15%, or at least about 20%, or at least about 25% antigen specific T cells. Further, these T cells generally display a memory phenotype (including both central and effector memory, as well as T memory stem cells). From the original sample isolated from the patient, the antigen-specific T cells in various embodiments are expanded (in about 7 days) from about 100-fold to about 10,000 fold, such as at least about 100-fold, or at least about 200-fold. After 2 weeks, antigen-specific T cells are expanded at least 1000-fold, or at least about 2000-fold, at least about 3,000 fold, at least about 4,000-fold, or at least about 5,000-fold in various embodiments. In some embodiments, antigen-specific T cells are expanded by greater than 5000-fold or greater than 10,000 fold after two weeks. After one or two weeks of expansion, at least about 10⁶, or at least about 10′, or at least about 10⁸, or at least about 10⁹ antigen-specific T cells are obtained.

Suitable incubation conditions (culture medium, temperature, etc.) include those used to culture T cells or T cell precursors, as well as those known in the art for inducing formation of antigen-specific T cells using DC or artificial antigen presenting cells.

The cell composition can be administered to patients by any appropriate routes, including intravenous infusion, intra-arterial administration, intralymphatic administration, and intratumoral administration.

In some embodiments, the patient receives immunotherapy with one or more checkpoint inhibitors, prior to (or optionally after) receiving the cell composition by adoptive transfer. In various embodiments, the checkpoint inhibitor(s) target one or more of CTLA-4 or PD-1/PD-L1, which may include antibodies against such targets, such as monoclonal antibodies, or portions thereof, or humanized or fully human versions thereof. In some embodiments, the checkpoint inhibitor therapy comprises ipilimumab or Keytruda (pembrolizumab).

In some embodiments, the patient receives about 1 to 5 rounds of adoptive immunotherapy (e.g., one, two, three, four or five rounds). In some embodiments, each administration of adoptive immunotherapy is conducted simultaneously with, or after (e.g., from about 1 day to about 1 week after), a round of checkpoint inhibitor therapy. In some embodiments, adoptive immunotherapy is provided about 1 day, about 2 days, about 3 days, about 4 days, about 5 days, about 6 days, or about 1 week after a checkpoint inhibitor dose. In some embodiments, the patient receives only a single administration of the cell composition.

In some aspects, the invention provides methods for personalized cancer immunotherapy. The methods are accomplished using the aAPCs to identify antigens to which the patient will respond, followed by administration of the appropriate peptide-loaded aAPC to the patient, or followed by enrichment and expansion of the antigen specific T cells ex vivo.

Genome-wide sequencing has dramatically altered our understanding of cancer biology. Sequencing of cancers has yielded important data regarding the molecular processes involved in the development of many human cancers. Driving mutations have been identified in key genes involved in pathways regulating three main cellular processes (1) cell fate, (2) cell survival and (3) genome maintenance. Vogelstein et al., Science 339, 1546-58 (2013).

Genome-wide sequencing also has the potential to revolutionize our approach to cancer immunotherapy. Sequencing data can provide information about both shared as well as personalized targets for cancer immunotherapy. In principle, mutant proteins are foreign to the immune system and are putative tumor-specific antigens. Indeed, sequencing efforts have defined hundred if not thousands of potentially relevant immune targets. Limited studies have shown that T cell responses against these neo-epitopes can be found in cancer patients or induced by cancer vaccines. However, the frequency of such responses against a particular cancer and the extent to which such responses are shared between patients are not well known. One of the main reasons for our limited understanding of tumor-specific immune responses is that current approaches for validating potential immunologically relevant targets are cumbersome and time consuming.

Although central tolerance abrogates T cell responses against self-proteins, oncogenic mutations induce neo-epitopes against which T cell responses can form. Mutation catalogues derived from whole exome sequencing provide a starting point for identifying such neo-epitopes. Using HLA binding prediction algorithms (Srivastava, PLoS One 4, e6094 (2009), it has been predicted that each cancer can have up 7-10 neo-epitopes. A similar approach estimated hundreds of tumor neo-epitopes. Such algorithms, however, may have low accuracy in predicting T cell responses, and only 10% of predicted HLA-binding epitopes are expected to bind in the context of HLA (Lundegaard C, Immunology 130, 309-18 (2010)). Thus, predicted epitopes must be validated for the existence of T cell responses against those potential neo-epitopes.

In certain embodiments, the nano-aAPC system is used to screen for neo-epitopes that induce a T cell response in a variety of cancers, or in a particular patient's cancer. Cancers may be genetically analyzed, for example, by whole exome-sequencing.

A list of candidate peptides can be generated from overlapping nine amino acid windows in mutated proteins. All nine-AA windows that contain a mutated amino acid, and 2 non-mutated “controls” from each protein will be selected. These candidate peptides will be assessed computationally for MHC binding using a consensus of MHC binding prediction algorithms, including Net MHC and stabilized matrix method (SMM). Nano-aAPC and MHC binding algorithms have been developed primarily for HLA-A2 allele. The sensitivity cut-off of the consensus prediction can be adjusted until a tractable number of mutation containing peptides (˜500) and non-mutated control peptides (˜50) are identified.

In an exemplary embodiments, the cell composition comprises, in a pharmaceutically acceptable carrier: at least 90% CD8+ T cells and less than 5% CD4+ T cells; at least 10⁶ CD8+ T cells specific for from 1 to 10 tumor-associated target peptide antigens, and CD8+ T cells specific for bacterial, viral, and/or fungal pathogens, wherein at least 30% of the CD8+ T cells are central memory and effector memory T cells with a ratio of from 25:75 to 75:25, with less than 10% of the CD8+ T cells being terminally differentiated T cells. In some embodiments, at least 50% of the CD8+ T cells specific for the tumor-associated target peptide antigens are central memory and effector memory T cells with a ratio of from 25:75 to 75:25, and with less than 10% of the CD8+ T cells being terminally differentiated T cells. In some embodiments, the cell composition further comprises from about 5% at about 20% T memory stem cells (T_{scm}), or from about 5% to about 15% T memory stem cells.

The cell composition further comprises a pharmaceutically acceptable carrier suitable for intravenous infusion, and which may be suitable as a cryoprotectant. In exemplary carrier is DMSO (e.g., about 10%). Cell compositions may be provided in unit vials or bags, and stored frozen until use. Unit doses may comprise from about 5×10⁵ to about 5×10⁶ cells per ml, in a volume of from 50 to 200 ml. In certain embodiments, the volume of the composition is <100 ml (e.g., from 50 to 100 ml).

In some aspects, the invention provides a method for treating a patient with cancer, comprising administering the cell composition described herein to a patient in need.

In some embodiments, the patient has a hematological cancer, which in some embodiments has relapsed after allogeneic stem cell transplantation. In some embodiments, the patient has acute myelogenous leukemia (AML) or myelodysplastic syndrome.

Other cancers that can be treated according to this disclosure include cancers that historically illicit poor immune responses or have a high rate of recurrence. Exemplary cancers include various types of solid tumors, including carcinomas, sarcomas, and lymphomas. In various embodiments the cancer is melanoma (including metastatic melanoma), colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, neuroblastoma, and glioma. In various embodiments, the cancer is stage I, stage II, stage III, or stage IV. In some embodiments, the cancer is metastatic and/or recurrent, and/or is nonresectable.

In some embodiments, the patient is refractory to chemotherapy and/or checkpoint inhibitor therapy.

In some embodiments, the patient further receives low dose cytokine therapy, which may improve the persistence and in vivo response.

In some embodiments, the cancer is a hematological malignancy, including leukemia, lymphoma, or myeloma. For example, the hematological malignancy may be acute myeloid leukemia, chronic myelogenous leukemia, childhood acute leukemia, non-Hodgkin's lymphomas, acute lymphocytic leukemia, chronic lymphocytic leukemia, myelodysplastic syndrome, malignant cutaneous T-cells, mycosis fungoids, non-MF cutaneous T-cell lymphoma, lymphomatoid papulosis, and T-cell rich cutaneous lymphoid hyperplasia. In an exemplary embodiment, the patient has a hematological cancer such as acute myelogenous leukemia (AML) or myelodysplastic syndrome, and in some embodiments the patient has relapsed after allogeneic stem cell transplantation. In some embodiments, the therapy does not induce GVHD.

In some embodiments, the patient, in addition to allogeneic stem cell transplantation, has also undergoes lympho-deleting therapy, cyto-reductive therapy, or immunomodulatory therapy (prior to administration of the cell therapy). In some embodiments, the cell therapy may be further provided with or without cytokine support post treatment.

In some embodiments, the patient has an infectious disease or is at risk for an infectious disease. For example, patients that have undergone HSCT are at particular risk for infectious disease, given the immunocompromised state. Infectious diseases that can be treated or prevented include those caused by bacteria, viruses, prions, fungi, parasites, helminths, etc. Such diseases include AIDS, hepatitis B/C, CMV infection, Epstein-Barr virus (EBV) infection, influenza, herpes virus infection (including shingles), and adenovirus infection. CMV, for example, is the most common viral pathogen found in organ transplant patients and is a major cause of morbidity and mortality in patients undergoing bone marrow or peripheral blood stem cell transplants. This is due to the immunocompromised status of these patients, which permits reactivation of latent virus in seropositive patients or opportunistic infection in seronegative individuals. In these embodiments, the patient may receive adoptive immunotherapy comprising T cells specific for pathogen antigens. The method can entail generation of virus-specific CTL derived from the patient or from an appropriate donor before initiation of the transplant procedure.

PTLD occurs in a significant fraction of transplant patients and results from Epstein-Barr virus (EBV) infection. EBV infection is believed to be present in approximately 90% of the adult population in the United States. Active viral replication and infection is kept in check by the immune system, but, as in cases of CMV, individuals immunocompromised by transplantation therapies lose the controlling T cell populations, which permits viral reactivation. This represents a serious impediment to transplant protocols. EBV may also be involved in tumor promotion in a variety of hematological and non-hematological cancers.

Other aspects and embodiments of the invention will be apparent to the skilled artisan.

### EXAMPLES

Antigen-specific T cells were enriched and expanded from donor cells isolated by leukapheresis. Cells were depleted of CD4+ cells by negative selection with CD4 microbeads. Resulting cells were enriched for antigen-specific T cells by incubating with paramagnetic nanoparticles (dextran-coated iron oxide nanoparticles, about 80-200 nm in diameter). The nanoparticles have dimeric HLA ligands conjugated to the surface (presenting the target peptide antigen), as well as an agonistic anti-CD28 monoclonal antibody. The dimeric HLA ligand contains two HLA-A2 domains, comprising the peptide binding clefts, each fused to an arm of the Ig hinge region. Dimeric HLA-Ig are co-expressed with β₂ microglobulin. Ligands and aAPC constructs are disclosed in WO 2016/044530 and WO 2016/105542, which are hereby incorporated by reference in their entirety.

Cells were incubated in the presence of the paramagnetic aAPC, then in the presence of a magnetic field for about 5 minutes. Cells associated with the particles were then recovered and expanded ex vivo for various lengths of time (generally from 1-2 weeks). Expansion was conducted in the presence of growth factors. For a two-week culture period, growth factors were added on days 1 and 7. Cells were re-stimulated with aAPCs on day 7.

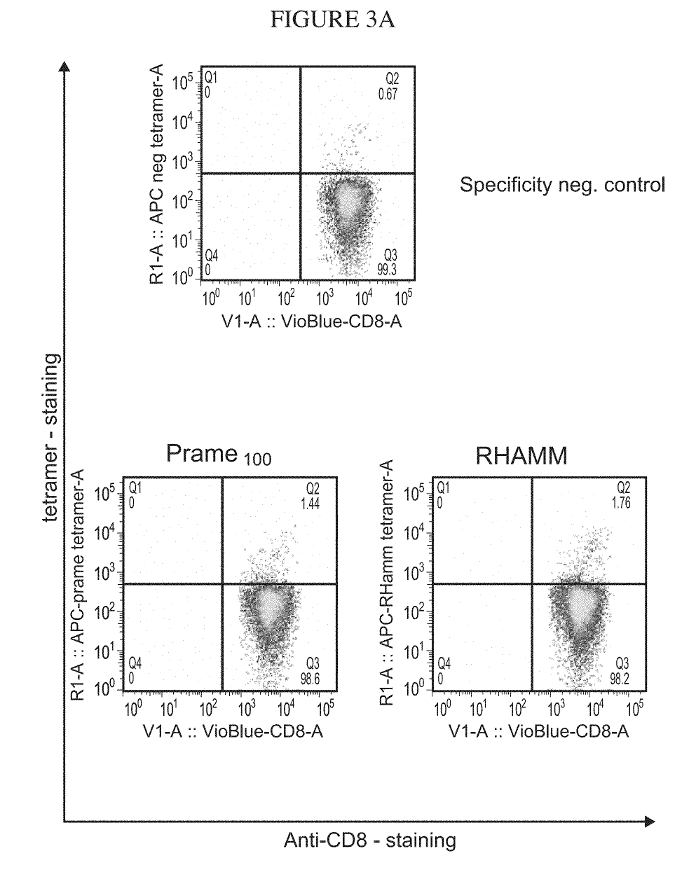

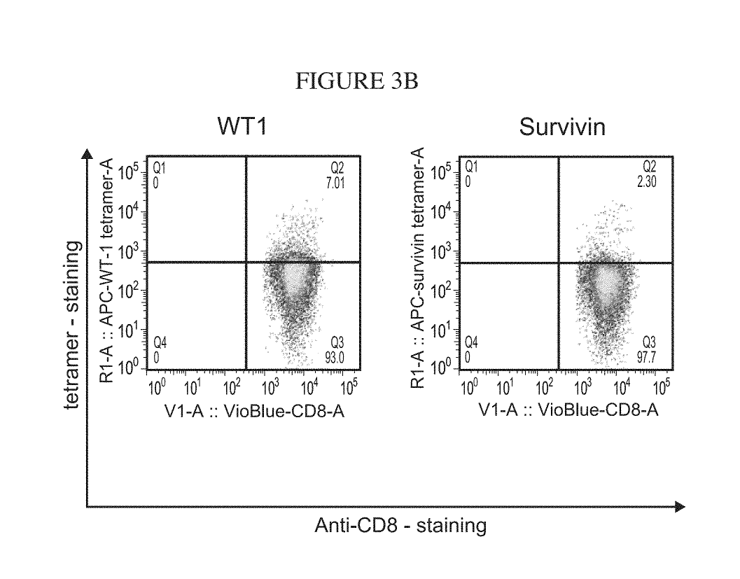

Antigen-specific T cells were also enriched and expanded in batch. For example, FIG. 3 shows batch enrichment and expansion of AML-specific peptides Prame100 RHAMM, WT1, and Survivin. At Day 7, the cells contain 1.4% specific for Prame, 1.8% specific for RHAMM, 7.0% specific for WT1, and 2.3% specific for Survivin. The total antigen-specific T cell component is 12.5% in this embodiment. T cells were characterized by tetramer staining.

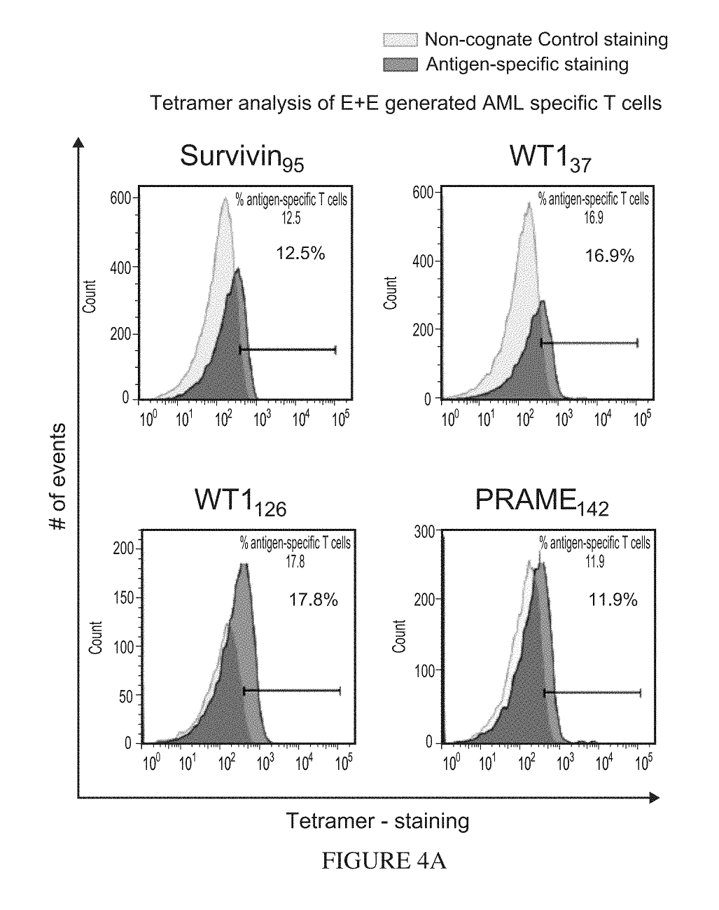

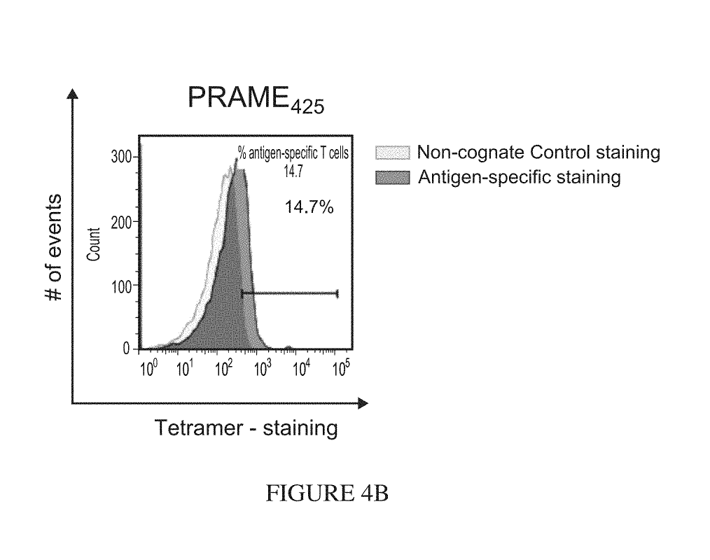

FIG. 4A and FIG. 4B show that the composition with individual stimulation and expansion for 2 weeks has consistent levels of AML antigen-specific T cells. Individual stimulation and expansion process consistently generates ˜15% antigen-specific T cells.

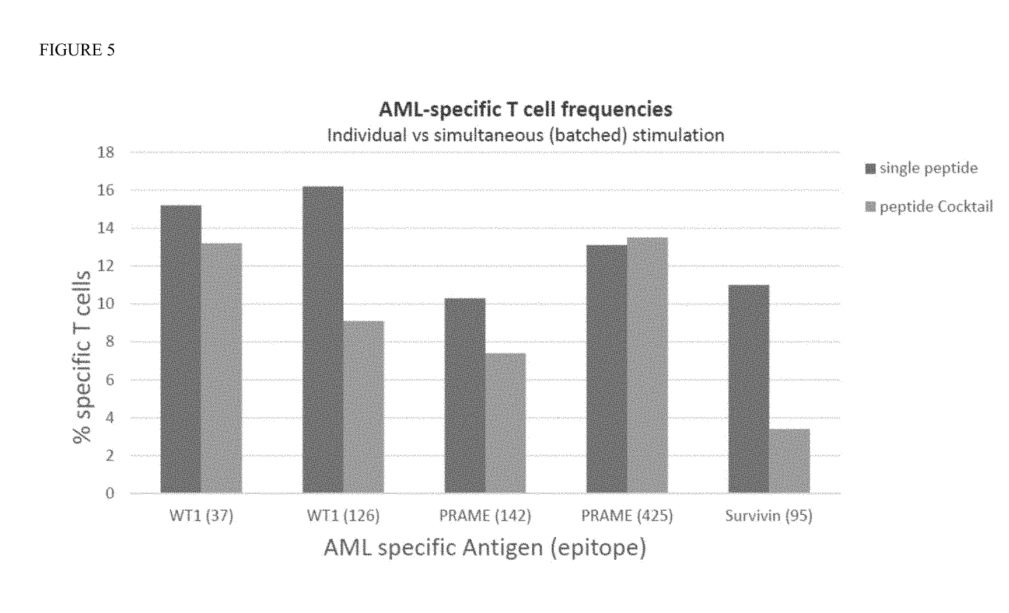

FIG. 5 shows that simultaneous stimulation/expansion process generates AML-specific T cell frequencies comparable to individual stimulation/expansion. The composition shown prepared by batch stimulation/expansion has ˜47% antigen-specific T cells.

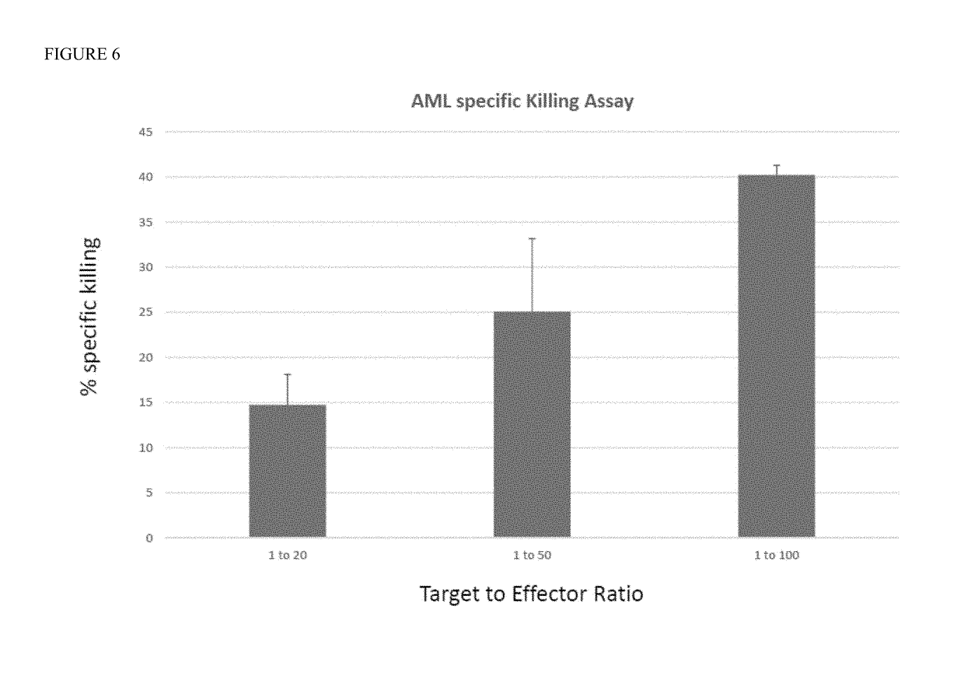

FIG. 6 shows that the generated T cells demonstrate antigen-specific killing of AML tumor cells (THP-1 cell line). AML specific T cells are directed at 5 epitopes from WT-1, PRAME, and Survivin. At 1 to 100 (Target to Effector ratio), ˜40% of target cells were killed.

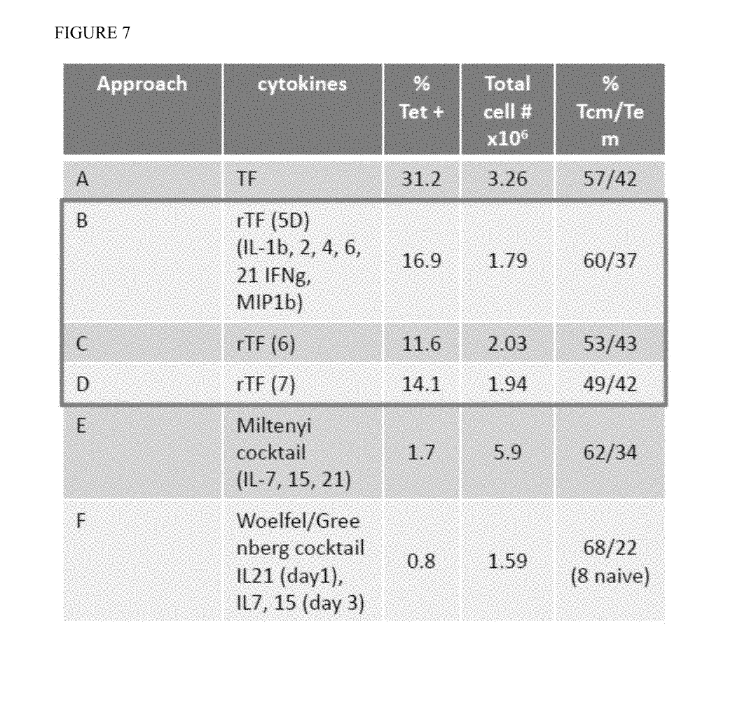

As shown in FIG. 7. the cytokine cocktail used for ex vivo expansion can impact the number and phenotype of resulting cells.

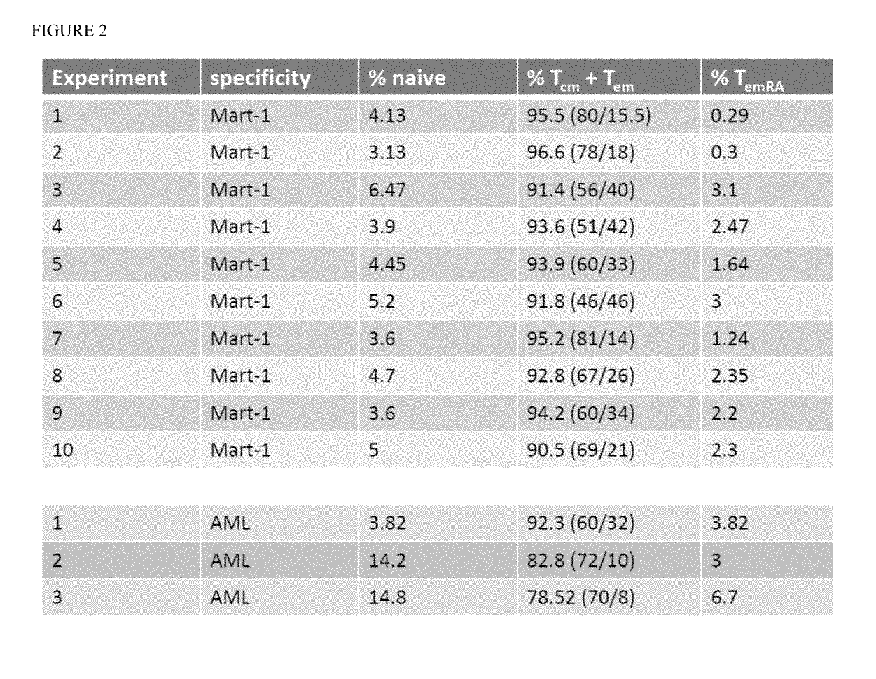

Cells were further characterized for their phenotype, either naive (CD62L+, CD45RA+), central memory (CD62L+, CD45RA−), effector memory (CD62L−, CD45RA−), and terminally differentiated memory (CD62L−, CD45RA+). MART-1 and AML specific T cells enriched and expanded ex vivo from donor lymphocytes are predominately central memory and effector memory phenotype. See FIG. 2. Particularly for AML peptides, in three representative experiments, naive cells were present at 3.82%, 14.2%, and 14.8%. Terminally differentiated memory cells were present at 3.82%, 3%, and 6.7%. Meanwhile, the central and effector memory component of the antigen-specific cells was 92.3%, 82.8%, and 78.52%.

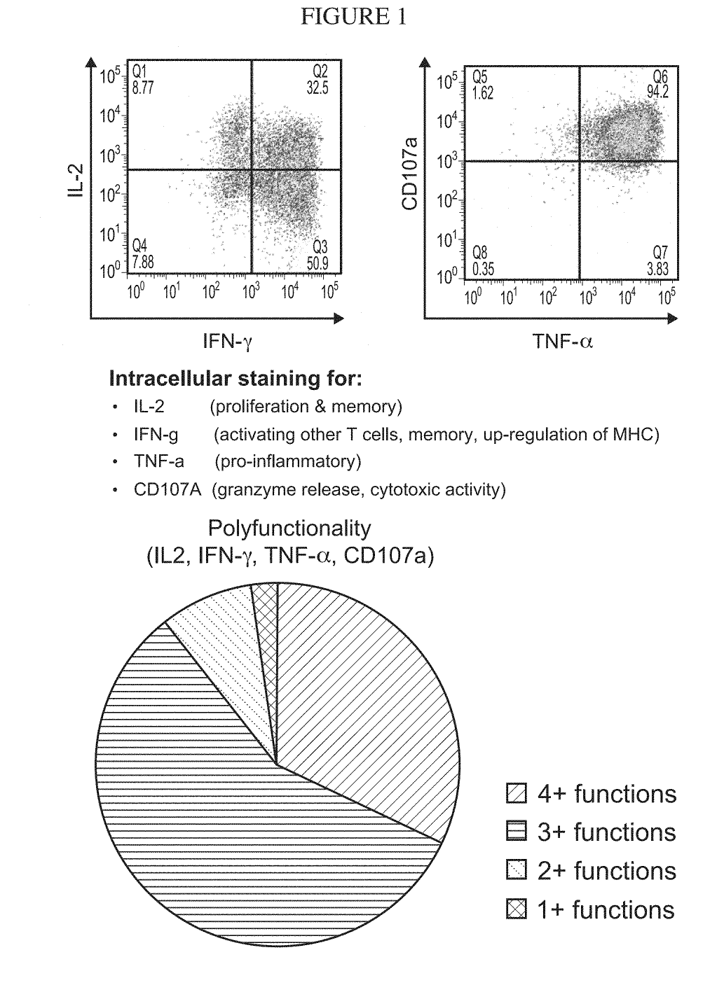

Cells were characterized by activation phenotype, namely, staining for IL-2 (proliferation and memory), IFN-γ (activating other T cells, memory, upregulation of MHC), TNF-α (pro-inflammatory), and CD107A (granzyme release, cytotoxic activity). See FIG. 1. As shown, the majority of cells have 3 or even 4 functions. For example, 32.5% of cells produce both IL-2 and IFN-γ upon activation, and 94.2% of the cells produce TNF-α and CD107a upon activation.

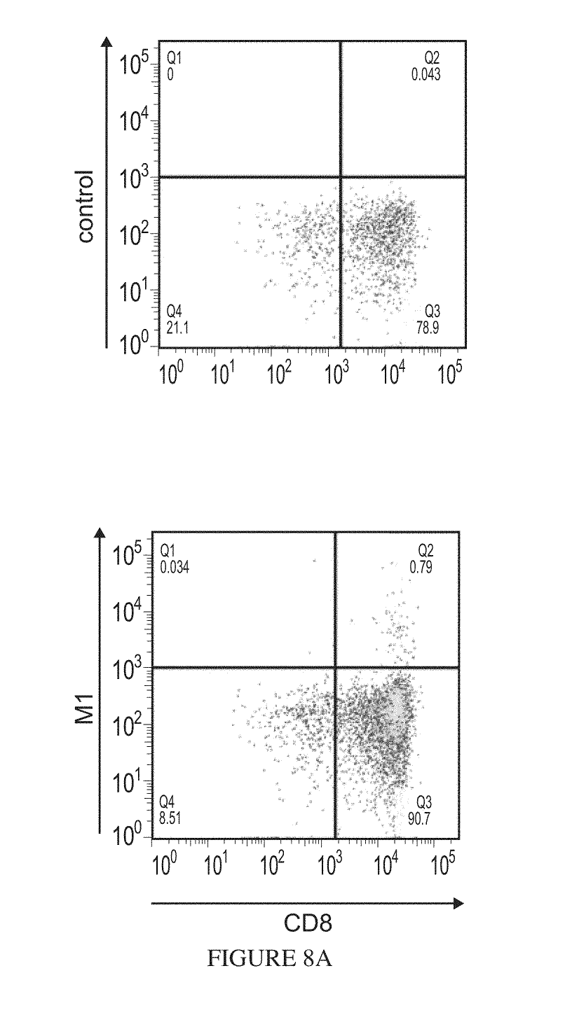

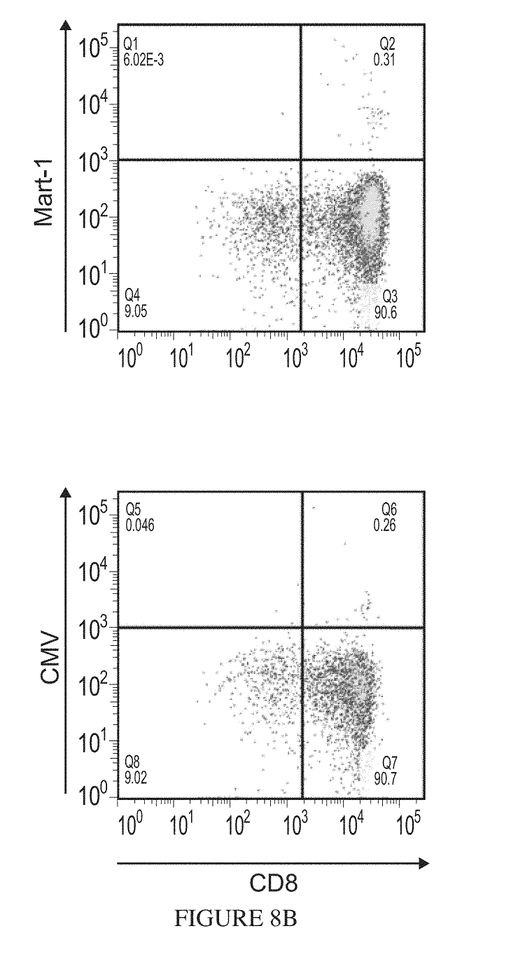

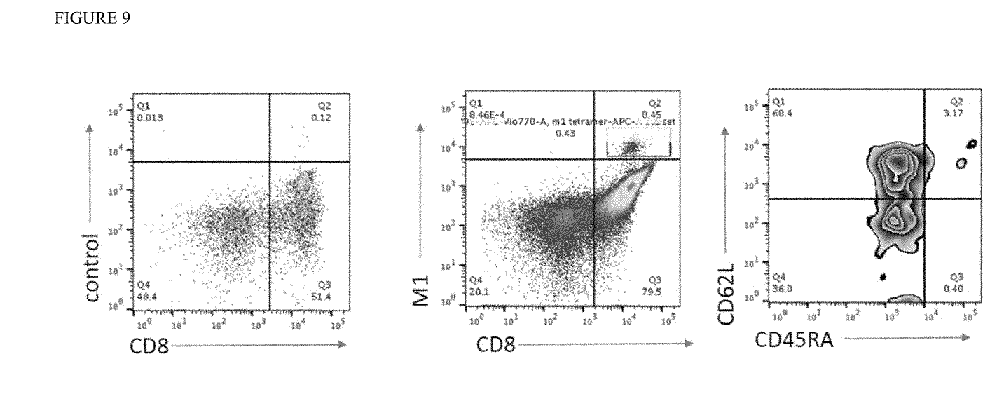

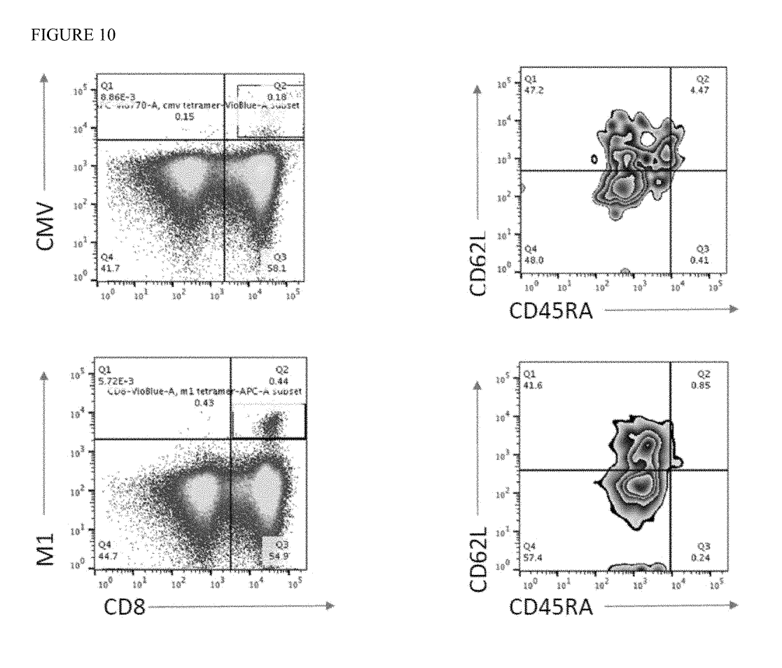

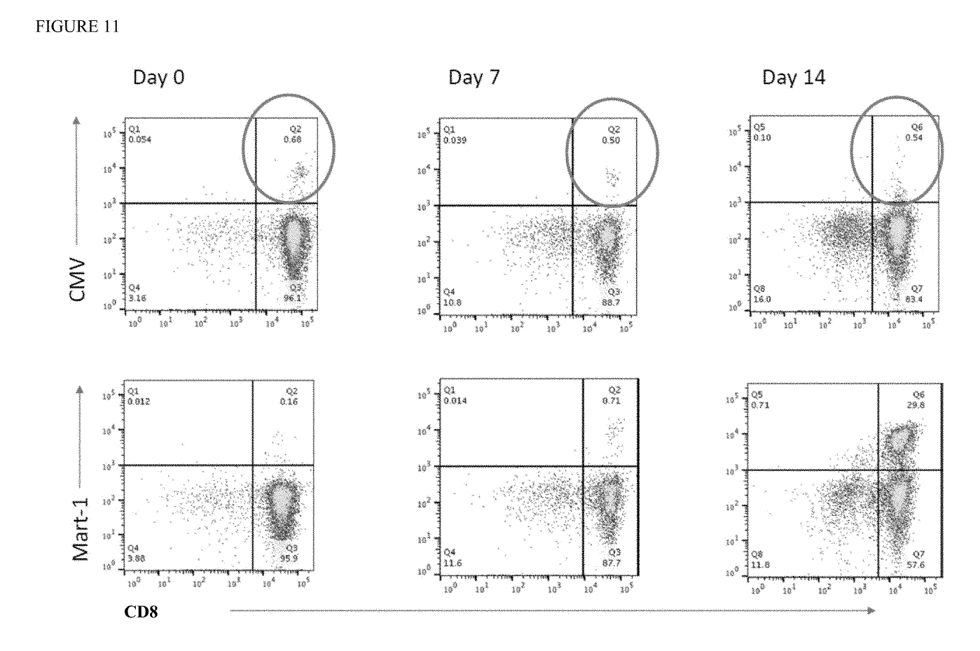

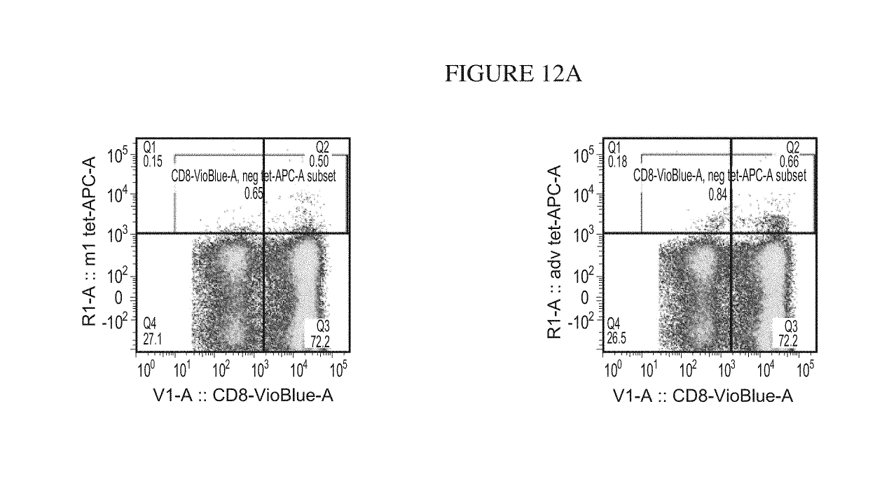

Bystander cells specific for viral antigens were further quantified by tetramer staining. FIG. 8 shows the presence of virus-specific bystander T cells on day 7 after MART-1-specific enrichment and expansion. FIG. 9 shows the presence of virus-specific bystander T cells on day 14 after MART-1-specific enrichment and expansion. These cells are also largely of central and effector memory phenotype. FIG. 10 shows the presence of virus-specific bystander T cells on day 14 after AML-specific enrichment and expansion. FIG. 11 shows detection of CMV-specific bystander T cells during MART-1 specific enrichment and expansion process. The percent of virus-specific bystander cells remains constant through Day 14 (between 0.5 and 1%), while the number and percent of MART-1 specific T cells rises dramatically.

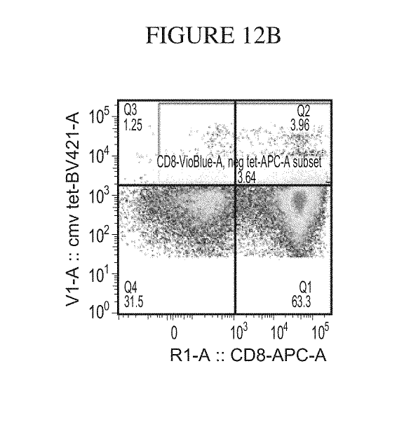

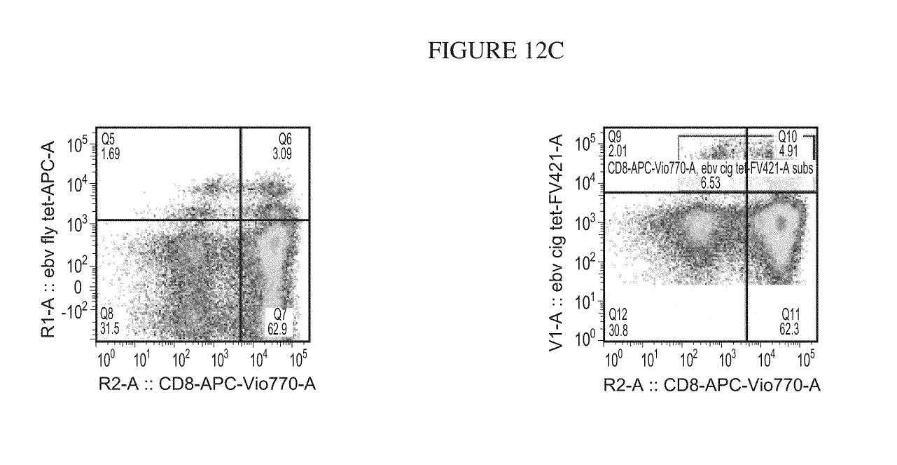

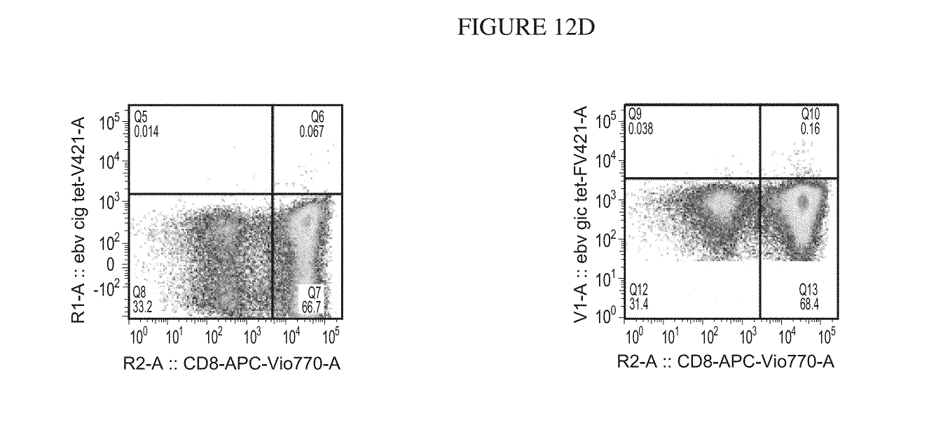

FIG. 12 shows detection of virus specific bystander cells on Day 14 after MART-1-specific enrichment and expansion using a recombinant T cell growth factor cocktail (IL-1β, IL-2, IL-4, IL-6, IL-21, IFN-γ, and MIP1-β), demonstrates maintenance and bystander expansion of viral specific T cells directed at multiple epitopes across Adeno, CMV, EBV and influenza.

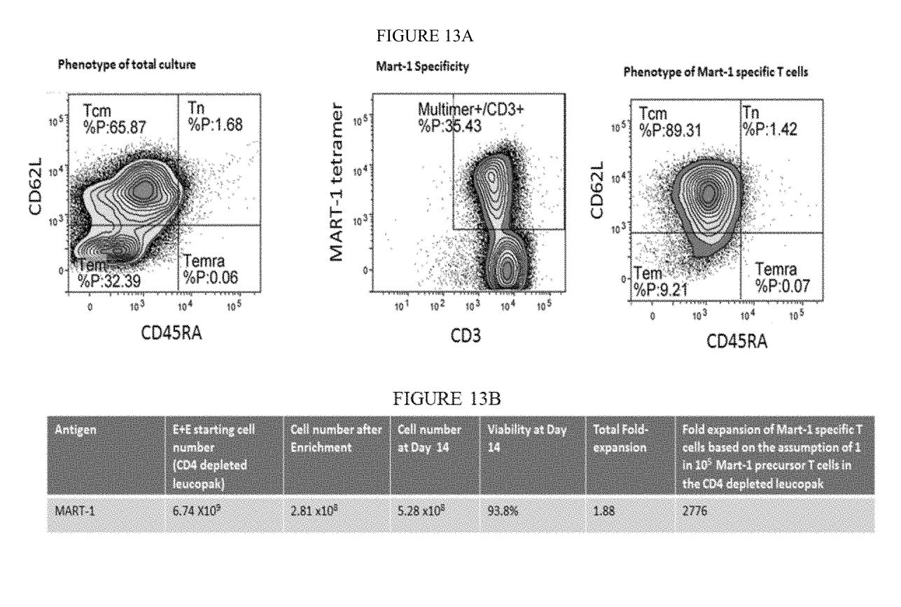

As shown in FIG. 13A and FIG. 13B, Mart-1 specific T cells were generated by the enrichment and expansion process, in the presence of the following cytokines during expansion: IL-2, IL-4, IL-6, IFN-γ, and IL1-β. The composition of this cytokine cocktail is shown in Table 1.

TABLE 1
Cytokine Cocktail for Expansion Phase

| | Specific Activity in final | Specific Activity in Stock |
|---|---|---|
| Cytokines | culture media (IU/ml) | Solution 50X (IU/ml) |
| IL-2 | 40 | 2000 |
| IL-4 | 2.5 | 125 |
| IL-6 | 50 | 2500 |
| IFNγ | 40 | 2000 |
| IL-1β | 15 | 750 |

In this experiment, 6.74×10⁹ CD8+ lymphocytes from a healthy donor were enriched as described above. After enrichment, there were 2.81×10⁸ total cells. At day 14 of expansion, there were 5.28×10⁸ total cells, showing a 1.88 fold expansion of total cells. These expanded cells at day 14 were ˜35% specific for MART-1, and about 94% viable (FIG. 13B). MART-1-specific cells were expanded about 2776 fold, assuming about 1 in 10⁵ precursor cells were MART-1 specific.

In evaluating the total culture, T cells had a phenotype of about 66% central memory and about 32% effector memory. Less than 2% of cells were naive, and the amount of T_{EMRA} cells were negligible. Further, MART-1 specific cells were about 89% central memory and about 9% effector memory, with less than 2% naive and a negligible number of T_{EMRA} cells.

1. An isolated cell composition suitable for adoptive immunotherapy, the composition comprising, in a pharmaceutically acceptable carrier: at least 10⁶ CD8+ T cells specific for one or more target peptide antigens, wherein at least 20% of T cells in the composition exhibit a central memory or effector memory phenotype.

2. The isolated cell composition of claim 1, wherein the CD8+ T cells are specific for from 1 to 100 target peptide antigens.

3. The isolated cell composition of claim 1, wherein T cell specificity toward a target peptide antigen in the composition is defined by WIC multimer staining.

4. The isolated cell composition of claim 1, wherein the target peptide antigens are tumor associated antigens.

5. (canceled)

6. The isolated cell composition of claim 1, wherein one or more target peptide antigens are bacterial, viral, fungal, or parasitic antigens.

7. The isolated cell composition of claim 1, comprising CD8+ T cells specific for at least one, two, three, four, or five target peptide antigens.

8. (canceled)

9. The isolated cell composition of claim 1, wherein the cell composition is at least 90% T cells.

10. (canceled)

11. The isolated cell composition of claim 1, wherein the cell composition is at least 5% CD8+ T cells specific for the target peptide antigens.

12-13. (canceled)

14. The isolated cell composition of claim 4, wherein the cell composition further comprises CD8+ T cells specific for bacterial, viral, and/or fungal pathogens.

15. The isolated cell composition of claim 14, wherein the CD8+ T cells specific for bacterial, viral, or fungal pathogens include T cells specific for antigens of influenza, CMV, EBV, and/or adenovirus.

16. The isolated cell composition of claim 1, wherein the T cells are at least 30% central and effector memory T cells.

17. The isolated cell composition of claim 16, wherein the T cells are at least 50% central and effector memory T cells.

18-19. (canceled)

20. The isolated cell composition of claim 16, wherein the T cells specific for the one or more target antigens are at least 50% central and effector memory T cells.

21-23. (canceled)

24. The isolated cell composition of claim 1, wherein the central and effector memory T cells are from 10:90 to 90:10 central to effector memory cells.

25-26. (canceled)

27. The isolated cell composition of claim 1, wherein the T cells are less than 20% terminally differentiated.

28-29. (canceled)

30. The isolated cell composition of claim 1, wherein the composition comprises less than 20% naive cells.

31-33. (canceled)

34. The isolated composition of claim 1, further comprising T memory stem cells.

35. (canceled)

36. The isolated cell composition of claim 1, wherein the CD8+ T cells display a polyfunctional phenotype upon activation.

37. The isolated cell composition of claim 1, wherein the cell composition is less than 10% CD4+ T cells.

38-41. (canceled)

42. The cell composition of claim 1, wherein the composition does not comprise T cells expressing a chimeric antigen receptor or a recombinant TCR.

43. The cell composition of claim 1, wherein the composition is produced by enrichment of CD8+ T cells specific for the target peptide antigens from source cells; and/or expansion of CD8+ T cells specific for the target peptide antigens from source cells.

44-50. (canceled)

51. The isolated cell composition of claim 43, wherein the antigen-specific T cells are enriched by aAPCs having an MHC class I ligand and optionally a co-stimulatory ligand.

52-53. (canceled)

54. The isolated cell composition of claim 43, wherein the enrichment is magnetic enrichment with paramagnetic aAPCs, and wherein the cells and aAPCs are incubated in the presence of a magnetic field for at least one minute.

55-57. (canceled)

58. The isolated cell composition of claim 43, wherein the enriched cells are expanded in culture for from 1 to 4 weeks.

59. The isolated cell composition of claim 58, wherein the cells are expanded in culture in the presence of one or more, two or more, or three or more of MIP-1β, IL-1β, IL-2, IL-4, IL-6, IL-7, IL-15, IL-21, and INF-γ, and IL-10.

60-61. (canceled)

62. The isolated cell composition of claim 59, wherein the cells are expanded in culture in the presence of at least one cytokine selected from MIP-1β, IL-1β, and IL-6, and IL-10.

63. The isolated cell composition of claim 59, wherein the cells are expanded in the presence of IL-4.

64. The isolated cell composition of claim 59, wherein the cells are expanded in the presence of IL-4 and IL-6.

65. The isolated cell composition of claim 59, wherein the cells are expanded in the presence of IL-4 and IL-1β.

66. The isolated cell composition of claim 59, wherein the cells are expanded in the presence of IL-4, IL-6, and IL-1β.

67. The isolated cell composition of claim 59, wherein the cells are expanded in the presence of IL-2, IL-4, and IL-6.

68. The isolated cell composition of claim 59, wherein the cells are expanded in culture in the presence of IL-2, IL-4, IL-6, INF-γ, and IL-1β, and IL-10.

69. (canceled)

70. The isolated cell composition of claim 1, wherein one or more target peptide antigens are selected from peptide epitopes of EBV, multiple myeloma, Survivin, WT-1, PRAME, Cyclin A1, and PR3.

71. An isolated cell composition suitable for adoptive immunotherapy, the composition comprising, in a pharmaceutically acceptable carrier:
at least 90% CD8+ T cells and less than 5% CD4+ T cells;
the CD8+ cells comprising at least 10⁶ CD8+ T cells specific for from 1 to 10 target peptide antigens, and CD8+ T cells specific for bacterial, viral, fungal and/or parasitic pathogens,
wherein at least 30% of the CD8+ T cells are central memory and effector memory T cells with a ratio of from 25:75 to 75:25, with less than 10% of the CD8+ T cells being terminally differentiated T cells and less than 10% of the CD8+ cells being naive cells; and
wherein at least 50% of the CD8+ T cells specific for the target peptide antigens are central memory and effector memory T cells with a ratio of from 25:75 to 75:25, are less than 10% terminally differentiated T cells, and are less than 10% naive cells.

72-74. (canceled)

75. The isolated cell composition of claim 71, further comprising from 5% to about 20% T memory stem cells.

76. The isolated cell composition of claim 71, wherein the target peptide antigens are tumor associated antigens, and are associated with hematological malignancy.

77. The isolated cell composition of claim 71, wherein one or more target peptide antigens are selected from peptide epitopes of EBV, multiple myeloma, Survivin, WT-1, PRAME, Cyclin A1, and PR3.

78. A method for treating a patient with cancer, comprising administering the cell composition of claim 1 to a patient in need.

79-83. (canceled)

84. A method for manufacturing the cell composition of claim 1, comprising:
depletion of CD4+ T cells from source cells;
enrichment of CD8+ T cells specific for the one or more target peptide, wherein the enrichment is magnetic enrichment with paramagnetic aAPCs; and
expansion of the CD8+ T cells ex vivo, wherein the CD8+ T cells are expanded in culture in the presence of one or more, two or more, or three or more of MIP-1β, IL-1β, IL-2, IL-4, IL-6, IL-7, IL-10, IL-12, IL-15, IL-21, and IFN-γ.


SEQUENCE LISTING (13 sequences)

SEQ ID NO: 1 - protein, 9 aa, Artificial Sequence
  Synthetic Polypeptide
RMFPNAPYL

SEQ ID NO: 2 - protein, 9 aa, Artificial Sequence
  Synthetic Polypeptide
CMTWNQMNL

SEQ ID NO: 3 - protein, 9 aa, Artificial Sequence
  Synthetic Polypeptide
VLDFAPPGA

SEQ ID NO: 4 - protein, 9 aa, Artificial Sequence
  Synthetic Polypeptide
SLGEQQYSV

SEQ ID NO: 5 - protein, 9 aa, Artificial Sequence
  Synthetic Polypeptide
VLDGLDVLL

SEQ ID NO: 6 - protein, 9 aa, Artificial Sequence
  Synthetic Polypeptide
NLTHVLYPV

SEQ ID NO: 7 - protein, 10 aa, Artificial Sequence
  Synthetic Polypeptide
SLYSFPEPEA

SEQ ID NO: 8 - protein, 10 aa, Artificial Sequence
  Synthetic Polypeptide
ALYVDSLFFL

SEQ ID NO: 9 - protein, 9 aa, Artificial Sequence
  Synthetic Polypeptide
SLLQHLIGL

SEQ ID NO: 10 - protein, 10 aa, Artificial Sequence
  Synthetic Polypeptide
ELTLGEFLKL

SEQ ID NO: 11 - protein, 10 aa, Artificial Sequence
  Synthetic Polypeptide
LDRERAKNKI

SEQ ID NO: 12 - protein, 9 aa, Artificial Sequence
  Synthetic Polypeptide
FLDRFLSCM

SEQ ID NO: 13 - protein, 11 aa, Artificial Sequence
  Synthetic Polypeptide
SLIAAAAFCLA